(19) United States
(12) Patent Application Publication
(10) Pub. No.: US 2024/0317895 A1
(43) Pub. Date: Sep. 26, 2024

(54) CARRIER FOR IMMOBILIZING PROTEIN AND PREPARATION METHOD THEREFOR

(71) Applicant: SOUTH CHINA UNIVERSITY OF TECHNOLOGY, Guangzhou (CN)

(72) Inventors: Zhanglin LIN, Guangzhou (CN); Yanrui YE, Guangzhou (CN); Xiaofeng YANG, Guangzhou (CN); Qiao LIN, Guangzhou (CN)

(73) Assignee: SOUTH CHINA UNIVERSITY OF TECHNOLOGY, Guangzhou (CN)

(21) Appl. No.: 18/009,578

(22) PCT Filed: Jun. 9, 2021
(86) PCT No.: PCT/CN2021/099144

§ 371 Date: Jun. 5, 2023

(30) Foreign Application Priority Data
Jun. 10, 2020 (CN) 202010525348.8

(51) Int. Cl. C07K 17/08; C07K 17/06

(52) U.S. Cl. CPC C07K 17/08; C07K 17/06

(57) ABSTRACT

A SpyCatcher peptide-modified carrier, a protein immobilizing carrier, and a protein immobilizing method. The method comprises: linking a SpyCatcher peptide to a carrier so as to obtain a SpyCatcher peptide-modified carrier; and then based on a SpyCatcher-SpyTag reaction, immobilizing a target protein containing SpyTag to the SpyCatcher-modified carrier. A protein immobilization process in which the method is used is fast, a condition is mild, no extra chemical reagent is needed, the immobilization efficiency and an activity recovery rate are high, and the homogeneity is good.

### TECHNICAL FIELD

The invention belongs to the field of bioengineering, in particular to the field of protein immobilization, for example, enzyme immobilization, and more particularly to a method of immobilizing a protein, a modified carrier for immobilizing a protein and a modified carrier with an immobilized protein.

### BACKGROUND ART

Compared with a free protein, an immobilized protein, such as an enzyme, is reusable and prone to be separated from a reaction system, and has an improved stability. Therefore, in applications involving biological binding or catalytic reaction of proteins, immobilized proteins have obvious advantages over free proteins.

Polyepoxy carriers, such as polypropylene and polystyrene, have been widely used in the immobilization of industrial enzymes for their high mechanical stability [Boller, Thomas, Christian Meier, and Stefan Menzler. “Eupergit oxirane acrylic beads: how to make enzymes fit for biocatalysis.” Organic Process Research & Development 6.4 (2002): 509-519; Mateo, Cesar, et al. “Immobilization of enzymes on heterofunctional epoxy supports.” Nature Protocols 2.5 (2007): 1022; McAuliffe, Joseph C. “Industrial enzymes and biocatalysis.” Handbook of industrial chemistry and biotechnology. Springer, Boston, M A, 2012. 1183-1227]. However, the recovery rate of activity of immobilized proteins on epoxy carriers are very low in most cases. Boller T et al. found from the statistics of 23 industrial enzymes immobilized with epoxy carriers that the recovery rates of their immobilized activity were mostly 30%-40% or lower [Boller, Thomas, Christian Meier, and Stefan Menzler. “Eupergit oxirane acrylic beads: how to make enzymes fit for biocatalysis.” Organic Process Research & Development 6.4 (2002): 509-519].

Epoxy-activated carriers (hereinafter referred to as epoxy carriers) can react with a variety of nucleophilic groups (e.g., lysine, cysteine, histidine, tyrosine and so on) on the surface of proteins through epoxy groups of the carriers and thus can realize multi-point covalent immobilization of proteins. Types and properties of epoxy carriers have great influences on the results of protein immobilization. For a same enzyme, there are usually great differences in immobilization efficiency and recovery rate of activity when different epoxy carriers are used [Seip, John E., et al. “Glyoxylic acid production using immobilized glycolate oxidase and catalase.” Bioorganic & medicinal chemistry 2.6 (1994): 371-378]. The physical properties of epoxy carriers make them highly suitable for industrial catalysis. Researchers have used several methods to improve epoxy carriers. Among them, the most common improvement strategy is to add other functional groups (e.g., amino, highly active disulfide bond, carboxyl, metal chelating groups and so on) onto an epoxy carrier. [Mateo, Cesar, et al. “Immobilization of enzymes on heterofunctional epoxy supports.” Nature Protocols 2.5 (2007): 1022]. Moreover, when modifying epoxy groups on epoxy carriers, the proportion of the epoxy groups modified also needs to be optimized. [Mateo, Cesar, et al. “Multifunctional epoxy supports: a new tool to improve the covalent immobilization of proteins. The promotion of physical adsorptions of proteins on the supports before their covalent linkage.” Biomacromolecules 1.4 (2000): 739-745]. Although such methods for modifying epoxy carriers may have obvious effects on some particular proteins, for different proteins, different modifications need to be made to epoxy carriers. Furthermore, the modification needs to be optimized, and its effect is difficult to predict. A lot of trial and error and screening works still need to be made for modifying epoxy carriers. To sum up, existing techniques of protein immobilization based on epoxy carriers are sensitive to the types of proteins and epoxy carriers, and require lots of epoxy carrier modification optimizations or epoxy carrier screening in order to obtain higher recovery rates of activity of immobilized proteins.

A directionally immobilized protein has a uniform orientation so that the activity of the protein can be maintained to the utmost extent. Therefore, the directional immobilization of proteins is widely used in the fields of industrial biocatalysis, clinical diagnosis, protein interaction analysis and the like [Wong, Lu Shin, Farid Khan, and Jason Micklefield. “Selective covalent protein immobilization: strategies and applications.” Chemical reviews 109.9 (2009): 4025-4053.; Wong, Lu Shin, Farid Khan, and Jason Micklefield. “Selective covalent protein immobilization: strategies and applications.” Chemical reviews 109.9 (2009): 4025-4053.; Meldal, Morten, and Sanne Schoffelen. “Recent advances in covalent, site-specific protein immobilization.” F1000Research 5 (2016).; Liese, Andreas, and Lutz Hilterhaus. “Evaluation of immobilized enzymes for industrial applications.” Chemical Society Reviews 42.15 (2013): 6236-6249]. Due to the presence of a plurality of same amino acids in most proteins, it is hard to realize directional immobilization of proteins by using traditional immobilization methods targeting specific amino acids (e.g., lysine, cysteine) [Mateo, Cesar, et al. “Immobilization of enzymes on heterofunctional epoxy supports.” Nature Protocols 2.5 (2007): 1022]. For example, by modifying oligoglycine on a carrier and catalyzing LPXTG motif at the C-terminus of a protein with sortase A, the protein can be linked to the N-terminus of the oligoglycine to realize directional immobilization of the protein; cysteine residues (C) on CXPXR may be modified with formylglycine generating enzyme (FGE) and then linked to amino groups of a carrier through Schiffs base reaction, thereby realizing directional immobilization of the protein [Popp, Maximilian W., et al. “Sortagging: a versatile method for protein labeling.” Nature chemical biology 3.11 (2007): 707-708; Raeeszadeh-Sarmazdeh, Maryam, Ranganath Parthasarathy, and Eric T. Boder. “Site-specific immobilization of protein layers on gold surfaces via orthogonal sortases.” Colloids and Surfaces B: Biointerfaces 128 (2015): 457-463; Rabuka, David, et al. “Site-specific chemical protein conjugation using genetically encoded aldehyde tags.” Nature protocols 7.6 (2012): 1052; Cho, Hwayoung, and Justyn Jaworski. “Enzyme directed formation of un-natural side-chains for covalent surface attachment of proteins.” Colloids and Surfaces B: Biointerfaces 122 (2014): 846-850]. The two methods mentioned above both require excessive enzymes for catalysis. Moreover, the rate of sortase A mediated immobilization is low, which is generally about 30% [Hata, Yuto, et al. “C-Terminal-oriented Immobilization of Enzymes Using Sortase A-mediated Technique.” Macromolecular bioscience 15.10 (2015): 1375-1380; Ito, Takaomi, et al. “Highly oriented recombinant glycosyltransferases: site-specific immobilization of unstable membrane proteins by using Staphylococcus aureus sortase A.” Biochemistry 49.11 (2010): 2604-2614].

### SUMMARY OF THE INVENTION

In the first aspect, there is provided a carrier modified by a SpyCatcher peptide, wherein, the carrier, when not modified by the SpyCatcher peptide, comprises a group capable of reacting with an amino group (NH₂), wherein the SpyCatcher peptide is covalently attached to the carrier through the reaction of the amino group with the group, and the SpyCatcher peptide linked to the carrier is capable of forming an isopeptide bond with a SpyTag peptide. In one embodiment, the group is selected from the group consisting of an epoxy group, an aldehyde group, an imide, a cyanate, an imidocarbonate, and a hydrazide group.

In one embodiment, the SpyCatcher peptide preferably comprises the amino acid sequence as set forth in SEQ ID NO: 21. In one embodiment, the SpyCatcher peptide comprises the amino acid sequence as set forth in SEQ ID NO: 1, 20, 22 or 23.

In one embodiment, the carrier is further linked to a fusion protein comprising a SpyTag peptide and a target protein, where the SpyCatcher peptide forms an isopeptide bond with the SpyTag peptide, and the isopeptide bond is formed by a Lys residue on the SpyCatcher peptide corresponding to Lys at position 56 of the amino acid sequence of SEQ ID NO: 1 with an Asp residue on the SpyTag peptide corresponding to Asp at position 7 of SEQ ID NO: 2. In one embodiment, the target protein is an enzyme. In one embodiment, the SpyTag peptide comprises the amino acid sequence of any of SEQ ID NOs: 2, 11-15 and 28. In one embodiment, the target protein is an enzyme, preferably selected from the group consisting of glutaryl-7-amidocephalosporanic acid acylase, glucose isomerase, nitrile hydratase, penicillin amidase, aspartase, fumarase, amino-acylase, lactase, aspartate-β-decarboxylase, and cephalosporin amidase.

In one embodiment, the carrier is made of an inorganic material, an organic material, or a composite material of an inorganic material and an organic material, where the inorganic material includes but not limited to silicon dioxide, metal oxides, and clay materials; and the organic material includes but not limited to agaroses, chitosans, alginates, gelatins, polyacrylic acids, polyacrylates, polystyrenes, polyamides, and polyacrylonitriles. Preferably, the carrier is an epoxy resin or an amino resin.

In one embodiment, there is provided an epoxy resin carrier modified by a SpyCatcher peptide, wherein the SpyCatcher peptide is covalently attached to the carrier through the reaction of an amino group with an epoxy group; and preferably, the epoxy resin carrier is made of a polyacrylate.

In one embodiment, the particle size of the epoxy resin is about 100-1000 m, and/or the content of epoxy groups of the epoxy resin is about 50-800 μmol/g wet.

In the second aspect, there is provided a method of preparing a carrier modified by a SpyCatcher peptide, comprising:
(1) providing a carrier comprising a group capable of reacting with an amino group (NH₂), wherein preferably the group is selected from the group consisting of an epoxy group, an aldehyde group, an imide, a cyanate, an imidocarbonate, and a hydrazide group;
(2) bringing a SpyCatcher peptide into contact with the carrier in a liquid environment; optionally, activating the group before the contact, and/or blocking the carrier after the contact; and
(3) optionally, isolating the carrier modified by the SpyCatcher peptide.

In the third aspect, there is provided a method of immobilizing a protein, comprising:
(1) providing a carrier modified by a SpyCatcher peptide according to the first aspect or a carrier modified by a SpyCatcher peptide obtained by the method according to the second aspect;
(2) providing a fusion protein comprising a SpyTag peptide and a target protein;
(3) bringing the carrier in step (1) into contact with the fusion protein in step (2) under a condition allowing the SpyCatcher peptide and the SpyTag peptide to form an isopeptide bond, and optionally, washing with a washing buffer to remove unimmobilized fusion proteins after the contact; and
(4) optionally, isolating the carrier linked to the fusion protein.

In one embodiment, the method can be used to obtain homogeneous carriers for enzyme immobilization.

In one embodiment, the SpyTag peptide comprises the amino acid sequence of SEQ ID NO: 21, 22, or 23. In one embodiment, the SpyTag peptide comprises the amino acid sequence of any of SEQ ID NOs: 2, 11-15, and 28.

In one embodiment, the step (1) comprises incubating the SpyCatcher peptide with a carrier not modified by the SpyCatcher peptide to obtain a carrier modified by the SpyCatcher peptide, and optionally, treating the carrier modified by the SpyCatcher peptide with a blocking buffer.

In one embodiment, the target protein is an enzyme, preferably selected from the group consisting of glutaryl-7-amidocephalosporanic acid acylase, glucose isomerase, nitrile hydratase, penicillin amidase, aspartase, fumarase, amino-acylase, lactase, aspartate-β-decarboxylase, and cephalosporin amidase.

In one embodiment, the carrier is selected from the group consisting of an epoxy resin and an amino resin, and is preferably made of an inorganic material, an organic material, or a composite material of an inorganic material and an organic material, wherein the inorganic material includes but not limited to silicon dioxide, metal oxides, and clay materials; or the carrier is made of an organic material, wherein the organic material includes but not limited to agaroses, chitosans, alginates, gelatins, polyacrylic acids, polyacrylates, polystyrenes, polyamides, and polyacrylonitriles.

In the fourth aspect, there is provided a kit comprising a carrier, a SpyCatcher peptide and optionally a fusion protein comprising a SpyTag peptide and a target protein, wherein the carrier and the SpyCatcher peptide are present separately or in the form of a carrier modified by a SpyCatcher peptide according to the first aspect. In one embodiment, the target protein is an enzyme.

### BRIEF DESCRIPTION OF THE DRAWINGS

FIG. 1: a schematic diagram of a method for specific immobilization of a protein based on SpyTag-SpyCatcher reaction.

FIG. 2: protein expression plasmid maps: a) a SpyCatcher expression plasmid map; b) a SpyTag-RFP expression plasmid map; and c) a SpyTag-GA expression plasmid map.

FIG. 3: expression and purification SDS-PAGE pictures of SpyCatcher, SpyTag-RFP, and SpyTag-GA. Lanes 1-5: protein quantitation standard samples containing bovine serum albumin (BSA), with loading concentrations in the order of 31.25 μg/mL, 62.5 μg/mL, 125 μg/mL, 250 μg/mL, 500 μg/mL. Lanes 6, 7, and 8 represent fusion proteins SpyTag-GA, SpyCatcher, and SpyTag-RFP, respectively.

FIG. 4: protein SDS-PAGE pictures before and after SpyCatcher immobilization with an epoxy carrier. Lanes 1-4: supernatants added with SpyCatcher by 5, 10, 20, and 40 mg/g of carrier before immobilization, respectively. Lanes 5-8: supernatants added with SpyCatcher by 5, 10, 20, and 40 mg/g of carrier after immobilization, respectively. Lanes 9-13: protein quantitation standard samples containing bovine serum albumin (BSA), with loading concentrations in the order of 31.25 μg/mL, 62.5 μg/mL, 125 μg/mL, 250 g/mL, 500 μg/mL.

FIG. 5: immobilized Spy-RFP. a) SDS-PAGE picture before and after immobilization of purified SpyTag-RFP, wherein Lane 1 represents SpyTag-RFP supernatant before immobilization; Lane 2 represents the supernatant after immobilization of SpyTag-RFP by the present method; Lane 3 represents the supernatant after immobilization of SpyTag-RFP in blocking control; Lane 4 represents the supernatant after immobilization of SpyTag-RFP by an epoxy immobilization method; and Lanes 5-9 represent protein quantitation standard samples containing bovine serum albumin (BSA), with loading concentrations in the order of 31.25 μg/mL, 62.5 μg/mL, 125 μg/mL, 250 μg/mL, 500 μg/mL. b) Comparison of carrier colors before and after immobilization of purified SpyTag-RFP, wherein No. 1 represents SpyTag-RFP immobilized by the present method; No. 2 represents SpyTag-RFP in the blocking control group; and No. 3 represents SpyTag-RFP immobilized by an epoxy method.

FIG. 6: direct immobilization of SpyTag-RFP from the supernatant of an E. coli cell lysis solution. a) SDS-PAGE picture before and after the immobilization of the supernatant of lysis of E. co/i overexpressing SpyTag-RFP, wherein Lane 1 represents the SpyTag-RFP cell lysis supernatant before immobilization; Lane 2 represents the cell lysis supernatant after immobilization of SpyTag-RFP by the present method; Lane 3 represents the cell lysis supernatant after immobilization of SpyTag-RFP in blocking control; Lane 4 represents the cell lysis supernatant after immobilization of SpyTag-RFP by an epoxy method; and Lanes 5-9 represent protein quantitation standard samples containing bovine serum albumin (BSA), with loading concentrations in the order of 31.25 μg/mL, 62.5 μg/mL, 125 μg/mL, 250 μg/mL, 500 μg/mL. b) fluorescence PAGE picture before and after immobilization of the supernatant of lysis of E. coli overexpressing SpyTag-RFP, where Lane 1 represents the SpyTag-RFP cell lysis supernatant before immobilization; Lane 2 represents the cell lysis supernatant after immobilization of SpyTag-RFP by the present method; Lane 3 represents the cell lysis supernatant after immobilization of SpyTag-RFP in blocking control; Lane 4 represents the cell lysis supernatant after immobilization of SpyTag-RFP by an epoxy immobilization method; and Lanes 5-9 represent protein quantitation standard samples containing bovine serum albumin (BSA), with loading concentrations in the order of 31.25 μg/mL, 62.5 μg/mL, 125 g/mL, 250 μg/mL, 500 μg/mL. c) a picture of comparison of carrier colors before and after immobilization of the supernatant of lysis of E. co/i overexpressing SpyTag-RFP, where No. 1 represents SpyTag-RFP immobilized by the present method; No. 2 represents SpyTag-RFP immobilized in a blocking control group; and No. 3 represents SpyTag-RFP immobilized by an epoxy method.

FIG. 7: SDS-PAGE pictures of the supernatants before and after immobilization of purified SpyTag-GA, wherein Lane 1 represents the SpyTag-GA supernatant before immobilization, Lane 2 represents the supernatant after immobilization of SpyTag-GA by the method of the invention, Lane 3 represents the supernatant after immobilization of SpyTag-GA in blocking control, Lane 4 represents the supernatant after immobilization of SpyTag-GA by an epoxy immobilization method, and Lanes 5-9 represent protein quantitation standard samples containing bovine serum albumin (BSA), with loading concentrations in the order of 31.25 μg/mL, 62.5 μg/mL, 125 μg/mL, 250 μg/mL, 500 μg/mL.

FIG. 8: SDS-PAGE picture of SpyTag-GA immobilized from the supernatant of E. co/i lysis solution. Lane 1 represents the SpyTag-GA cell lysis supernatant before immobilization. Lane 2 represents the cell lysis supernatant after immobilization of SpyTag-GA by the present method. Lane 3 represents the cell lysis supernatant after immobilization of SpyTag-GA in blocking control. Lane 4 represents the cell lysis supernatant after immobilization of SpyTag-GA by an epoxy immobilization method. Lanes 5-9 represent protein quantitation standard samples containing bovine serum albumin (BSA), with loading concentrations in the order of 31.25 μg/mL, 62.5 μg/mL, 125 μg/mL, 250 μg/mL, 500 μg/mL.

FIG. 9: comparison of immobilization efficiency of an immobilized protein with a SpyCatcher modified carrier obtained by the method of the invention and an epoxy carrier.

FIG. 10: comparison of immobilization efficiency of an immobilized protein with a same carrier by the present method and an epoxy method. a) LX1000-EP; b) LX-107S; c) LX-103B; d) HFA001; e) ECR8204; f) ECR8285.

FIG. 11: comparison of recovery rate of activity of an immobilized protein with a SpyCatcher modified carrier obtained by the present method and an epoxy carrier.

FIG. 12: comparison of recovery rate of activity of an immobilized protein with a same carrier by the present method and an epoxy method. a) LX1000-EP; b) LX-107S; c) LX-103B; d) HFA001; e) ECR8204; f) ECR8285.

FIG. 13: comparison of relative activity of an immobilized protein with a SpyCatcher modified carrier obtained by the present method and an epoxy carrier.

FIG. 14: comparison of relative activity of an immobilized protein with a same carrier by the present method and an epoxy method.

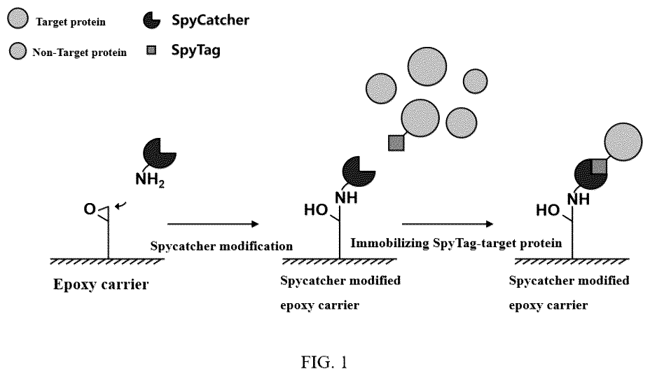

### DETAILED DESCRIPTION OF THE INVENTION

The present invention provides a method of immobilizing a protein to solve the technical problems of being sensitive to types of proteins and carriers and needing to carry out a trial and error experiment on immobilization and carrier screening in protein immobilization.

In the first aspect, the present invention provides a carrier modified by a SpyCatcher peptide, wherein the carrier, when not modified by the SpyCatcher peptide, comprises a group capable of reacting with an amino group (NH₂), wherein the SpyCatcher peptide is covalently attached to the carrier through reaction of the amino group with the group, and the SpyCatcher peptide linked to the carrier is capable of forming an isopeptide bond with a SpyTag peptide.

As used herein, the carrier is any carrier that can be used to immobilize a protein as long as it contains a group capable of reacting with an amino group (NH₂). The carrier may be made of various materials, including but not limited to inorganic materials, organic materials, or composite materials of inorganic materials and organic materials. In one embodiment, the inorganic materials include but not limited to diatomite, kaolinite, silica gels, porous glasses, activated carbon, calcium carbonate, ceramics, silicon dioxide, metal oxides, and clay materials. In one embodiment, the organic materials include but not limited to agarose, chitosan, alginates, gelatins, polyacrylic acids, polyacrylates, polymethylacrylic acids, polymethylacrylates, polystyrenes, polyamides, and polyacrylonitriles. In one preferred embodiment, the carrier is an epoxy resin carrier or an amino resin carrier. In one particularly preferred embodiment, the carrier is a carrier made of a polyacrylic acid (a polyacrylate), particularly an epoxy resin carrier.

As used herein, the group capable of reacting with an amino group (NH₂) refers to a group capable of reacting with an amino group in the presence or absence of an activator. It is known in the art how to activate a group so as to react with an amino group (e.g., see Hermanson, Greg T. Bioconjugate techniques. Academic press, 2013). The amino group may be an amino group at the N-terminus of a peptide, or an amino group on a side chain of an amino acid residue, for example, ε-amino group of Lys.

The SpyCatcher peptide is attached to the carrier through the reaction of one or more amino groups with the group.

In one embodiment, the SpyCatcher peptide is attached to the carrier through the reaction of one amino group with the group. In one embodiment, the SpyCatcher peptide is attached to the carrier through the reaction of the N-terminal amino group with the group. In another embodiment, the SpyCatcher peptide is attached to the carrier through the reaction of the ε-amino group of one Lys with the group.

In one embodiment, the SpyCatcher peptide is attached at multiple sites to the carrier through the reaction of a plurality of (i.e., two or more) amino groups with the group. The two or more (e.g., 2, 3, 4, 5, 6, 7, 8, 9) amino groups may be the N-terminal amino group and the ε-amino groups of one or more Lys, or the ε-amino groups of two or more Lys.

In one embodiment, the group capable of reacting with an amino group (NH₂) is selected from the group consisting of an epoxy group, an aldehyde group, an imide, a cyanate, an imidocarbonate, and a hydrazide group.

It has been found in recent years that the protein ligase of the fibronectin-binding protein (FbaB) in Streptococcus pyogenes is capable of rapidly forming an isopeptide bond between the aspartate at position 117 (Asp117, corresponding to Asp at position 7 of SEQ ID NO: 2 as disclosed herein) and the lysine at position 31 (Lys31, corresponding to Lys at position 56 of SEQ ID NO: 1 as disclosed herein). This reaction is referred to as Spy chemistry. After FbaB is divided into two parts, SpyCatcher and SpyTag, and the SpyCatcher and the SpyTag can react with each other and thus are linked through an isopeptide bond to form a protein. The Asp117 and Lys31 residues described above are very important for the Spy reaction. For example, if the ε-NH₂ of Lys31 has been reacted with other groups, the Spy reaction cannot occur. See, for example Science, 2007, 318, 1625-1628] [PNAS, 2012; 109:690; Angew. Chem. Int. Ed. 2010, 49, 8421-8425, JACS. 2011, 133, 478-485.

The Spy chemistry has been widely studied in the fields of protein labeling, protein binding, protein topology, protein materials, etc. (PNAS, 2012; 109:690; Bioconjugate Chem. 2017, 28: 1544-1551; J. Am. Chem. Soc. 2013, 135: 13988-13997, Angew. Chem. Int. Ed. 2019, 58, 11097-11104; Angew. Chem. Int. Ed. 2014, 53: 6101-6104; PNAS, 2014, 111, 31, 11269-11274, Matter 1, 1-17). The Spy reaction of SpyTag and SpyCatcher is highly specific, and both SpyTag (13 amino acids) and SpyCatcher (115 amino acids) are small. Moreover, SpyCatcher may be obtained by conventional recombinant expression techniques (e.g., using Escherichia coli as expression host, 110-150 mg of His tag purified SpyCatcher protein may be obtained per liter of fermentation broth).

The present invention provides a method of immobilizing a protein based on the Spy chemistry, comprising: obtaining a SpyCatcher modified carrier by linking the SpyCatcher to a group of the carrier through a covalent bond, and immobilizing a fusion protein of SpyTag and a target protein to the SpyCatcher modified carrier by the Spy chemistry. The inventors surprisingly found that after a SpyCatcher peptide reacts with a carrier, Lys31 can still form an isopeptide bond, i.e., undergo the Spy reaction, with Asp117.

As used herein, the terms “protein”, “peptide”, “polypeptide” and “amino acid sequence” can be used interchangeably, and refer to a polymer of any length, for example, two or more amino acid residues. The term further includes an amino acid polymer modified naturally or artificially, for example, forming a disulfide bond, glycosylation, esterification, acetylation, phosphorylation, or any other operations and modifications, such as conjugation with a tag or a bioactive component. Conventional single-letter or three-letter amino acid residue codes are used herein.

As used herein, the term “amino acid” or “aa” refers to natural and synthetic amino acids, and amino acid analogues and amino acid mimics that function in a similar way to natural amino acids. Natural amino acids refer to amino acids encoded by genetic codons and those amino acids modified later, such as hydroxyproline, γ-carboxyl glutamate, and O-phosphoserine. Amino acid analogues refer to compounds having a substantially same chemical structure (i.e., α-C binding to hydrogen, a carboxyl group, an amino group and a R group) as natural amino acids. Amino acid mimics refer to chemical compounds that have a structure different from the general chemical structure of amino acids but function in a similar way to natural amino acids.

As used herein, the SpyCatcher peptide is any peptide or its variants that are capable of reacting with a SpyTag peptide or its variants to form an isopeptide bond linkage (Spy reaction). Various SpyCatcher peptides and variants thereof are known in the art, e.g., see Zakeri, B., Fierer, J. O., Celik, E., Chittock, E. C., Schwarz-Linek, U., Moy, V. T., & Howarth, M. (2012). Peptide tag forming a rapid covalent bond to a protein, through engineering a bacterial adhesin. Proceedings of the National Academy of Sciences, 109(12), E690-E697; Keeble, A. H., Banerjee, A., Ferla, M. P., Reddington, S. C., Anuar, I. N. K., & Howarth, M. (2017). Evolving accelerated amidation by SpyTag/SpyCatcher to analyze membrane dynamics. Angewandte Chemie International Edition, 56(52), 16521-16525; Keeble, A. H., Turkki, P., Stokes, S., Anuar, I. N. K., Rahikainen, R., Hytonen, V. P., & Howarth, M. (2019). Approaching infinite affinity through engineering of peptide-protein interaction. Proceedings of the National Academy of Sciences, 116(52), 26523-26533.

In one embodiment, the SpyCatcher peptide comprises the amino acid sequence of SEQ ID NO: 21 or an amino acid sequence thereof having one or more amino acid substitutions. In one embodiment, the substitution is an amino acid substitution at one or more of the positions 15, 40, 67, 69, 75, 81, 83, 86, 91 of SEQ ID NO: 21. In one embodiment, the substitution is one or more substitutions selected from the group consisting of K15R, Q40H, A67P, T69E, Q75D, N81D, K83E, K86E and I91T.

In one embodiment, the SpyCatcher peptide comprises the amino acid sequence of SEQ ID NO: 21, for example, the amino acid sequence of SEQ ID NO: 1 or 20.

In one embodiment, the SpyCatcher peptide comprises the amino acid sequence of SEQ ID NO: 20 and an amino acid substitution at one or more of the positions 2, 9, 13, 19, 37, 62, 89, 91, 97, 103, 105, 108 and 113 of the amino acid sequence of SEQ ID NO: 20. In one embodiment, the one or more amino acid substitutions are selected from the amino acid substitutions D2T, S9G, Q13P, I19T, K37R, Q62H, A89P, T91E, Q97D, N103D, K105E, K108E, and I113T at one or more positions in the amino acid sequence of SEQ ID NO:20. In one embodiment, the one or more amino acid substitutions are selected from the amino acid substitutions D2T, S9G, Q13P, I19T, K37R, Q62H, K105E, and 1113T at one or more positions in the amino acid sequence of SEQ ID NO:20. In one embodiment, the SpyCatcher peptide comprises the amino acid sequence of SEQ ID NO: 22 or 23.

In one embodiment, the SpyCatcher peptide having one or more amino acid substitutions according to the present invention may comprise an amino acid sequence having at least 80%, at least 85%, at least 90%, at least 95%, at least 96%, at least 97%, at least 98%, at least 99% or higher sequence identity to the amino acid sequence of SEQ ID NO: 1, 20, 21, 22, or 23.

Sequence identity may be determined by a commercially available computer program that calculates the identity percentage between two or more sequences using any appropriate algorithm, for example, using default parameters. A typical example of such a computer program is CLUSTAL. More advantageously, BLAST algorithm is used with parameters being set to default values. A detail introduction to the BLAST algorithm can be found on website National Center for Biotechnology Information (NCBI).

In one embodiment, the carrier is an epoxy carrier or an amino carrier, i.e. an epoxy group or an amino group present on the carrier, such as an epoxy resin and an amino resin. Due to the chemical activity of epoxy groups, the ring of an epoxy group may be opened by using a variety of compounds containing reactive hydrogen. In a covalent carrier, an epoxy carrier is almost an ideal matrix and is capable of immobilizing a protein very easily on the laboratory scale and the industrial scale. The epoxy carrier reacts with the protein under a very mild experiment condition (e.g., pH 7.0). In one embodiment, the carrier is a carrier made of an inorganic material, an organic material, or a composite material of an inorganic material and an organic material, where the inorganic material includes but not limited to silicon dioxide, metal oxides, and clay materials; and the organic material includes but not limited to agaroses, chitosans, alginates, gelatins, polyacrylic acids, polyacrylates, polymethylacrylic acids, polymethylacrylates, polystyrenes, polyamides, and polyacrylonitriles.

As used herein, the epoxy resin refers to an epoxy group containing resin, and the amino resin refers to an amino group containing resin, where the epoxy group and the amino group may be an epoxy group and an amino group obtained by modification on the resins.

As used herein, the epoxy resin refers to an epoxy group containing polymer which may include, for example, an epoxy resin, an amino epoxy resin, a carboxyl epoxy resin, and a mercapto-disulfide bond epoxy group. The epoxy resin, for example, includes but not limited to Lifetech™ ECR8285, ECR8204, ECR8209, LX1000EA, LX1000EP, LX103B, EP200, LX1000HFA, HFA001, LX107S, LX1000SW, LX1000SD, Eupergit® C, Eupergit® C250L, Sepabead® FP-EC3, Sepabead® EC-EP/M, Sepabeads® EC-Ep, ES1, ES103, ES105, ES108 and ES109.

In one embodiment, the epoxy resin has a particle size of about 5-2000 μm, for example, about 10-2000, 20-2000, 30-2000, 40-2000, 50-2000, 50-1900, 50-1800, 50-1700, 50-1600, 50-1500, 50-1400, 50-1300, 50-1200, 50-1100, 50-1000, 50-900, 50-800, 50-700, 50-600, 50-500, 50-450, 50-400, 50-350, 100-450, 100-400, 100-350, 100-300, 150-450, 150-400, 150-350, 150-300 μm, such as about 5, 10, 20, 30, 40, 50, 60, 70, 80, 90, 100, 110, 120, 130, 140, 150, 160, 170, 180, 190, 200,210, 220, 230, 240, 250, 260, 270, 280, 290, 300, 350, 400, 450, 500, 600, 700, 800, 900, 1000, 1100, 1200, 1300, 1400, 1500, 1600, 1700, 1800, 1900, 2000 μm. As used herein, the particle size refers to the average diameter (median (D50)) of particles, denoted by μm.

In one embodiment, the epoxy resin has an epoxy group content of less than 1000 μmol/g wet, such as about 10-1000 μmol/g wet, for example, about 10-900, 10-800, 10-700, 10-600, 10-500, 10-400, 10-300, 10-200, 10-150, 10-100, 20-900, 20-800, 20-700, 20-600, 20-500, 20-400, 20-300, 20-200, 20-150, 20-100, 30-900, 30-800, 30-700, 30-600, 30-500, 30-400, 30-300, 30-200, 30-150, 30-100, 40-900, 40-800, 40-700, 40-600, 40-500, 40-400, 40-300, 40-200, 40-150, 40-100, 50-900, 50-800, 50-700, 50-600, 50-500, 50-400, 50-300, 50-200, 50-150, 50-100, 50-90, 50-80, 70-90, 80-100, 75-95, 390-520, 585-780 gmol/g wet, such as about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950, 1000 gmol/g wet. As used herein, the epoxy group content refers to the mole number of epoxy groups per gram of wet resin, denoted by gmol/g wet.

In one embodiment, the epoxy resin is hydrophilic.

In one embodiment, the epoxy resin is made of a polyacrylate, such as polymethylacrylate, polyethylacrylate, polypropylacrylate, polybutylacrylate, polyisobutylacrylate, and poly(tert-butylacrylate). In one embodiment, the epoxy resin is made of a polymethylacrylate. In one embodiment, the epoxy resin is made of a polyethylacrylate. In one embodiment, the epoxy resin is made of a polypropylacrylate. In one embodiment, the epoxy resin is made of a polybutylacrylate. In one embodiment, the epoxy resin is made of a polyisobutylacrylate. In one embodiment, the epoxy resin is made of a poly(tert-butylacrylate).

In one embodiment, the group on the epoxy resin that is linked to the Spycatcher peptide is an epoxy group.

In one embodiment, there is provided an epoxy resin carrier modified by a SpyCatcher peptide as described herein, wherein the SpyCatcher peptide is covalently attached to the carrier through the reaction of an amino group with an epoxy group of the resin. Preferably, the epoxy resin carrier is made of a polyacrylate. More preferably, the particle size of the epoxy resin is about 100-350 μm, and/or the epoxy group content of the epoxy resin is about 50-100 gmol/g wet.

As used herein, the amino (NH₂) resin refers to an amino group containing polymer which includes e.g. an amino group containing polyacrylate, an amino group containing polymethylacrylate, an amino group containing polystyrene, etc. The amino resin includes but not limited to LX-1000EA, LX-1000HA, ECR8305, ECR8309, ECR8315, ECR8404, ECR8409, and ECR8415.

In the second aspect, there is provided a method of preparing a carrier modified by a SpyCatcher peptide, comprising:
(1) providing an unmodified carrier containing a group capable of reacting with an amino group (NH₂), wherein the group is preferably selected from the group consisting of an epoxy group, an aldehyde group, an imide, a cyanate, an imidocarbonate, and a hydrazide group;
(2) bringing a SpyCatcher peptide into contact with the carrier in a liquid environment; optionally, activating the group before the contact, and/or blocking the carrier after the contact; and
(3) optionally, isolating the carrier modified by the SpyCatcher peptide.

The carrier, the SpyCatcher peptide and the like are as described above.

The unmodified carrier described herein may be obtained in any way known in the art. Before the contact with the SpyCatcher peptide, the carrier may be activated by using an activator, or may be directly brought into contact with the SpyCatcher peptide (not using the activator). Reaction conditions are known in the art or may be determined easily according to techniques known in the art.

As used herein, the liquid environment refers to a liquid environment suitable for the SpyCatcher peptide to be covalently attached to the carrier through the reaction of an amino group (e.g., amino groups on the N-terminus and side chain, or an amino group on side chain) on the peptide with the group capable of reacting with the amino group, for example, PBS buffer solution (e.g., 0.1 M, pH 7.0). For example, see Zakeri, Bijan, et al. “Peptide tag forming a rapid covalent bond to a protein, through engineering a bacterial adhesin.” Proceedings of the National Academy of Sciences 109.12 (2012): E690-E697.

As used herein, the activation means treating a group so that the group can react with an amino group on a SpyCatcher peptide, e.g. using glutaraldehyde as the activator to activate an amino group on a carrier. For example, see Walt, David R., and Venetka I. Agayn. “The chemistry of enzyme and protein immobilization with glutaraldehyde.” (1994): 425-430; Immobilization of enzymes and cells. Humana Press, 2006.

In one embodiment, the step (2) of the method in the second aspect comprises: incubating the SpyCatcher peptide with an unmodified carrier, for example, for at least about 1, 2, 3, 4, 5, 6, 7, 8, 9, 10, 11, 12, 24, 36, 48 hours, to obtain a carrier modified by the SpyCatcher peptide; and optionally, treating the carrier modified by the SpyCatcher peptide with a blocking buffer solution, for example, for at least about 1, 2, 3, 4, 5, 6, 7, 8, 9, 10, 11, 12, 24, 36, 48 hours. In one embodiment, the incubation is carried out in a centrifugal reaction at a speed of about 20-50 rpm, for example, about 25-40, 25-35, or 25-30 rpm. After the completion of blocking, the modified carrier may be isolated, e.g. removing the blocking buffer solution by centrifugation. In one embodiment, the ratio of the SpyCatcher peptide and the unmodified carrier for incubation is 10-40 mg of peptide/g of carrier, for example, 10, 15, 20, 25, 30, 35, 40 mg of peptide/g of carrier, particularly preferably 20 mg of peptide/g of carrier.

Step (2) of the method in the second aspect may be carried out at any appropriate temperature and pH. For example, the temperature may be about 20-37° C., such as about 20-35° C., 25-37° C., 25-35° C., 20-30° C., or 25-35° C., particularly about 20, 21, 22, 23, 24, 25, 26, 27, 28, 29, 30, 31, 32, 33, 34, 35, 36, 37° C. The pH value may be any appropriate pH, for example about 4.0-10.0, such as about 5.0-9.0, 6.0-8.0, or 6.0-7.0, particularly about 4.0, 4.5, 5.0, 5.5, 6.0, 6.5, 7.0, 7.5, 8.0, 8.5, 9.0, 9.5, 10.0.

As used herein, the term “blocking” means completely reacting the remaining groups (e.g., epoxy groups) that has not reacted with SpyCatcher for the purpose of preventing the groups from reacting with the target protein in the immobilization process of the second step. The blocking buffer used herein is a blocking buffer commonly used in the art. Blocking buffer solutions suitable for the groups mentioned herein are known in the art (e.g., see Hermanson, Greg T. Bioconjugate techniques. Academic press, 2013; Immobilization of enzymes and cells. Humana Press, 2006). For example, the blocking buffer used herein is an epoxy group blocking buffer commonly used in the art, such as glycine, ethanolamine, and bovine serum albumin. In one embodiment, the blocking buffer includes but not limited to: glycine, pH 8.5; and ethanolamine, pH 8.5. In one embodiment, the blocking buffer solution is 3M glycine at pH 8.5.

The isolation of the carrier modified by the SpyCatcher peptide in step (3) of the method in the second aspect may be performed by using any appropriate technique known in the art, for example, centrifugation.

In a further embodiment, a carrier modified by a SpyCatcher peptide described herein or obtained by the method described herein is linked to a fusion protein including a SpyTag peptide and a target protein, wherein the SpyCatcher peptide and the SpyTag peptide form an isopeptide bond (the Spy reaction), thereby linking the target protein to the carrier modified by the SpyCatcher peptide.

As described above, the SpyTag peptide is also any peptide that is known in the art and capable of undergoing the Spy reaction. Various techniques and means for generating fusion proteins are known in the art. In a fusion protein, the SpyTag peptide may be located at the N-terminus, the C-terminus or at any position in the middle of the fusion protein as long as the obtained fusion protein has the desired functional activity. It is easy for a person skilled in the art to detect the functional activity of a protein according to the technical knowledge in the art, which is within the technical capabilities of skilled persons in the art.

In one embodiment, the SpyTag peptide described herein comprises the amino acid sequence of any of SEQ ID NO: 2, 11, 12, 13, 14, 15 or 28.

In one embodiment, the SpyTag peptide is located at the N-terminus or the C-terminus of the fusion protein.

In one embodiment, the SpyTag peptide and the target protein in the fusion protein described herein may be linked by a linker, e.g. SpyTag-linker-target protein or target protein-linker-SpyTag.

As used herein, the linker is a peptide or other molecule that links the SpyTag peptide to the target protein. The linking may be achieved by any method of linking two moieties known in the art as long as the linker does not obviously compromise the desired functional activity of the protein in the fusion protein and/or does not obviously compromise the Spy reaction of the SpyTag peptide and the SpyCatcher peptide. It is easy for a person skilled in the art to determine and select an appropriate linker according to the technical knowledge in the art.

As used herein, the fusion protein of a SpyTag peptide and a target protein described herein includes the SpyTag peptide and the target protein, and may also include a polypeptide linker and an affinity tag. In one embodiment, the polypeptide linker is located between the SpyTag and the target protein.

As used herein, “affinity tag” refers to an affinity tag added to the N-terminus or the C-terminus of the fusion protein to facilitate the subsequent purification of the target protein. The fusion protein of a SpyTag and a target protein may be a purified fusion protein or unpurified fusion protein, such as a cell lysis solution. In one embodiment, the amino acid sequence of the SpyTag is set forth in SEQ ID NO: 2. The SpyTag peptide may be at the C-terminus or the N-terminus of the target protein.

In one embodiment, the linker has a length of preferably not less than 9 amino acid residues, for example, 9, 11, 13, 15 amino acid residues or longer. Any linker known to a person skilled in the art may be used in the present invention. The linker may be a peptide. Typical amino acid residues for use as linkers are glycine, seine, tyrosine, cysteine, lysine, glutamate, aspartate, etc. Examples of such linker include but not limited to (GGGGS)₃ (SEQ ID NO:27), (G)ₙS(G)ₙ, where n=4, 5, 6, or 7. In one embodiment, the linker is (GGGGS)₃.

Herein, the target protein linked to SpyTag may be any protein desired to be immobilized on a carrier, including but not limited to, for example, enzymes, cofactors, chaperonins, etc. In one embodiment, the target protein is an enzyme, for example, any enzyme known in the art that may be immobilized on a carrier described herein, and the enzyme may be of any appropriate source, which may be isolated from natural sources such as bacteria or artificially synthesized, for example, expressed by recombinant technology.

In one embodiment, the enzyme is selected from the group consisting of glucose isomerase (EC 5.3.1.5), nitrile hydratase (EC 4.2.1.84), penicillin amidase (EC 3.5.1.11), aspartase (EC 4.3.1.1), fumarase (EC 4.2.1.2), amino-acylase (EC 3.5.1.14), lactase (EC 3.2.1.108), aspartate-β-decarboxylase (EC 4.1.1.12), and cephalosporin amidase (EC 3.5.1.11).

In one embodiment, the present disclosure provides an epoxy resin carrier modified by a SpyCatcher peptide, wherein the SpyCatcher peptide is covalently attached to the carrier through reaction of an amino group with an epoxy group on the carrier. In one embodiment, the SpyCatcher peptide has the amino acid sequence of SEQ ID NO: 1. In one embodiment, the epoxy resin carrier is made of a polyacrylate. Preferably, the particle size of the epoxy resin is about 100-350 m, and/or the epoxy group content of the epoxy resin is about 50-100 μmol/g wet.

In one embodiment, the present disclosure provides an epoxy resin carrier with an immobilized protein. The epoxy resin is linked to a SpyCatcher peptide having the amino acid sequence of SEQ ID NO: 1, and the protein is a fusion protein including a SpyTag peptide having the amino acid sequence of SEQ ID NO: 2 and a target protein. Preferably, the epoxy resin is made of a polyacrylate.

In the third aspect, the present disclosure provides a method of immobilizing a protein, comprising:
(1) providing a carrier modified by a SpyCatcher peptide according to the first aspect or a carrier modified by a SpyCatcher peptide obtained by the method according to the second aspect;
(2) providing a fusion protein including a SpyTag peptide and a target protein; and
(3) bringing the carrier in step (1) into contact with the fusion protein in step (2) under a condition allowing the SpyCatcher peptide and the SpyTag peptide to form an isopeptide bond, and optionally, washing with a washing buffer solution to remove unimmobilized fusion proteins after the contact; and
(4) optionally, isolating the carrier linked to the fusion protein.

The carrier, the SpyCatcher peptide, the SpyTag peptide, the protein and the like are as described above.

In one embodiment, the step (1) of the method in the third aspect comprises incubating the SpyCatcher peptide with an unmodified carrier, for example, for at least about 1, 2, 3, 4, 5, 6, 7, 8, 9, 10, 11, 12, 24, 36, 48 hours, to obtain a carrier modified by the SpyCatcher peptide, and optionally, treating the carrier modified by the SpyCatcher peptide with a blocking buffer solution, for example, for at least about 1, 2, 3, 4, 5, 6, 7, 8, 9, 10, 11, 12, 24, 36, 48 hours. In one embodiment, the incubation is carried out in a centrifugal reaction at a speed of about 20-50 rpm, for example, about 25-40, 25-35, or 25-30 rpm. After the completion of blocking, the modified carrier may be isolated, e.g. removing the blocking buffer solution by centrifugation. In one embodiment, the ratio of the SpyCatcher peptide and the unmodified carrier for the incubation is 10-40 mg of peptide/g of carrier, for example, 10, 15, 20, 25, 30, 35, 40 mg of peptide/g of carrier, particularly preferably 20 mg of peptide/g of carrier.

Step (1) of the method in the third aspect can be carried out at any appropriate temperature and pH. For example, the temperature may be about 20-37° C., such as about 20-35° C., 25-37° C., 25-35° C., 20-30° C. or 25-35° C., particularly about 20, 21, 22, 23, 24, 25, 26, 27, 28, 29, 30, 31, 32, 33, 34, 35, 36, 37° C. The pH value may be any appropriate pH, for example about 4.0-10.0, such as about 5.0-9.0, 6.0-8.0, or 6.0-7.0, particularly about 4.0, 4.5, 5.0, 5.5, 6.0, 6.5, 7.0, 7.5, 8.0, 8.5, 9.0, 9.5, 10.0.

The target protein linked to the SpyTag peptide in step (2) of the method in the third aspect may be an isolated and purified protein, for example, a fusion protein isolated and purified by a known technique in the art, and may also be a cell lysis solution that contains the fusion protein of the peptide and the target protein and is not further purified, for example, a lysis solution of cells expressing the fusion protein.

The fusion protein may be obtained by recombinant expression in a prokaryote, a yeast, or a higher eukaryote cell. Exemplified prokaryotes include bacteria of Escherichia, Bacillus, Salmonella, Pseudomonas, and Streptomyces. In a preferred embodiment, recombinant cells are Escherichia cells, preferably Escherichia coli. In a specific embodiment of the present invention, the recombinant cells used are Escherichia coli BL21 (DE3) cells (Novagen). The target protein described in the present invention may be any protein, such as red fluorescence protein (RFP) and glutaryl-7-amidocephalosporanic acid acylase (GA) illustrated in the present examples.

The cell lysis solution may be obtained by a known method in the art, for example, methods of cell disruption include but not limited to: ultrasonication, homogenization, high pressure (e.g., in a French cell press), osmolysis, detergents, lyases, organic solvents, or combinations thereof. In one embodiment, the disruption is carried out under the first pH condition (i.e., weak basic pH, for example, pH 7.2-8.5, preferably pH 7.4) such that the cell membranes of host cells are disrupted and a protein supernatant is released from the disrupted cells and still keeps soluble. The purified fusion protein may be obtained by affinity tag purification. In some embodiments, the affinity tag is a commonly used 6×His tag located at the C-terminus of the target protein. The released protein supernatant is purified by protein affinity chromatography. After the cell membranes of recombinant cells are disrupted, by centrifugation, the supernatant may be collected and insoluble precipitates are removed. Affinity chromatography purification is carried out by means of the His tag of the fusion protein.

In step (3) of the method in the third aspect, the modified carrier and the fusion protein are brought into contact under a condition of allowing the SpyCatcher peptide and the SpyTag peptide to form an isopeptide bond. The condition of allowing the SpyCatcher peptide and the SpyTag peptide to form an isopeptide bond (undergo the Spy reaction) (as described above) is known in the art.

As used herein, the contact means physical association of the modified carrier with the protein, for example, mixing two substances in a solution, or adding one substance (e.g., the modified carrier) to a solution containing another substance (e.g., the fusion protein).

In one embodiment, step (3) of the method in the third aspect comprises bringing the SpyCatcher modified carrier into contact with the fusion protein, for example, for at least about 1, 2, 3, 4, 5, 6, 7, 8, 9, 10, 11, 12, 24, 36, 48 hour. In one embodiment, the contact is carried out in a centrifugal reaction at a speed of about 20-50 rpm, for example, about 25-40, 25-35, or 25-30 rpm.

Step (3) of the method in the third aspect may be carried out at any appropriate temperature and pH. For example, the temperature may be about 4-37° C., such as about 5-35° C., 10-35° C., 15-35° C., 20-35° C., 25-37° C., 25-35° C., 20-30° C., or 25-35° C., particularly about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37° C. The pH value may be any appropriate pH, for example about 4.0-10.0, such as about 5.0-9.0, 6.0-8.0, or 6.0-7.0, particularly about 4.0, 4.5, 5.0, 5.5, 6.0, 6.5, 7.0, 7.5, 8.0, 8.5, 9.0, 9.5, 10.0. In one embodiment, pH 6.0-7.0 is particularly preferred.

In one embodiment, step (3) of the method in the third aspect comprises incubating the fusion protein of a SpyTag peptide and a target protein with the carrier linked to a SpyCatcher peptide obtained in step (1), and optionally, washing with the washing buffer solution to remove unimmobilized fusion proteins.

As used herein, the washing buffer solution is a protein washing buffer solution commonly used in the art, such as a phosphate buffer solution (PBS) and Tris-HCl buffer solution.

The isolation of the carrier linked to the fusion protein in step (4) of the method in the third aspect may be performed by using any appropriate technique known in the art, for example, centrifugation.

In one embodiment, there is provided a method of immobilizing a protein based on Spy reaction, said method comprising:
incubating a SpyCatcher peptide with an epoxy carrier to obtain a carrier modified by the SpyCatcher peptide;
treating the carrier modified by the SpyCatcher peptide with a blocking buffer solution;
incubating a fusion protein of a SpyTag peptide and a target protein with the carrier modified by the SpyCatcher peptide;
washing with a washing buffer solution to remove unimmobilized fusion proteins; and
optionally, isolating the carrier linked to the fusion protein.

In the fourth aspect, there is provided a kit comprising a carrier, a SpyCatcher peptide, and optionally a fusion protein of a SpyTag peptide and a target protein.

The carrier, the SpyCatcher peptide, the SpyTag peptide, the protein and the like are as described above.

In one embodiment, the carrier and the SpyCatcher peptide are present separately. Before use, the carrier and the peptide may be brought into contact (e.g., mixed in a solution) to prepare a carrier modified by a SpyCatcher peptide according to the first aspect.

In another embodiment, the carrier and the SpyCatcher peptide are present in the form of a carrier modified by a SpyCatcher peptide according to the first aspect.

Unless indicated otherwise in the context, the word “or” is intended to include “and”.

As used herein, “optional” or “optionally” means that an event or situation described subsequently occur or does not occur, and this description includes the cases in which the event or situation occurs and does not occur. For example, an optional step means that the step is present or absent.

As used herein, the term “about” refers to a numerical range including the specific numerical values, and a person skilled in the art can reasonably consider it as being similar to the specific numerical value. In some embodiments, the term “about” means falling within a standard error of measurement generally accepted in the art. For example, in some embodiments, the term “about” means +/−10% or 5% of the specific numerical value.

As used herein, when specific numerical values or ratios are listed for a feature in the description, ranges composed of any two numerical values or ratios are also covered. For example, when numerical values 1, 2, 3, and 4 are listed, ranges 1-2, 1-3, 1-4, 2-3, 2-4 and 3-4 are also covered.

The method provided in the present invention is compatible with existing commercial immobilizing carriers without need of new carrier surface chemistry and of extra enzyme catalysts such as sortase, and a cell lysis solution containing a desired protein may be directly used for immobilization. The immobilization process of the present method (in the case of an epoxy carrier) takes only one step, and is characterized by specific linking reaction, high efficiency, mild conditions (room temperature, neutral pH), without need of extra chemical reagents. The immobilization carriers improved by the present method has good generality, high immobilization efficiency and recovery rate of activity, and good homogeneity, which requires less screening work of immobilization carriers.

### DESCRIPTION OF SPECIFIC EMBODIMENTS

The specific embodiments of the present invention will be further described below by way of the examples, but the embodiments of the present invention are not limited thereto. Methods used in the following examples are conventional methods unless otherwise specified. See, for example, [symbol] Molecular Cloning: A Laboratory Manual [symbol] (Sambrook, J., Russell, David W., Molecular Cloning: A Laboratory Manual, 3^{rd} edition, 2001, NY, Cold Spring Harbor) for specific steps. All primers and sequencing were synthesized by Sangon Biotech (Shanghai) Co., Ltd.

### Example 1: Construction of Protein Expression Plasmids

Oligonucleotide primers used in the present invention were as shown in Table 1.

TABLE 1
Oligonucleotide Primer Sequences Used in the
Present Invention

| | | SEQ |
|---|---|---|
| | | ID |
| Primer | Nucleotide sequence (5′-3′)^{ª} | NO |
| SpyCatcher-F | ATTCCATATGCCGACCCCACCGACCACGCC | 3 |
| SpyCatcher-R | GTGGTGGTGGTGGTGGTGCTCGAGAATGTG | 4 |
| | CGCATCGCCTTTGGT | |
| RFP-F | ATATACATATGGCGCATATTGTGATGGTGG | 5 |
| | ATGCGTATAAACCGACCAAAGGCGGTGG | |
| RFP-R | GGGGGGAAGCTTTTTATACAGTTCGTCCAT | 6 |
| | AC | |
| GA-F | GCGAGCTCGAACCGACCAGCACCCCGCAGG | 7 |
| | CGCCGATCGC | |
| GA-R | GCAAGCTTCGGTTTAAAGTTAAACGGGGTG | 8 |
| | CGTTC | |

^{a}The underlined parts of the primer were recognition sites of restriction enzymes Nde I, Hind III, Sac I, and Xho I.

### 1.1 Construction of Expression Plasmid of SpyCatcher

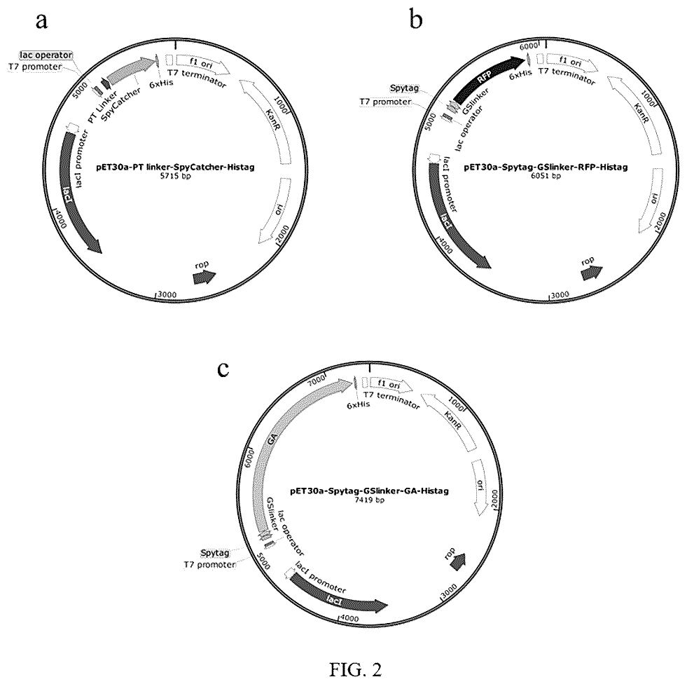

The expression plasmid pET30a(+)-PT linker-SpyCatcher-His (SEQ ID NO: 17) constructed in this example was used to express the protein Ptlingker-SpyCatcher-His, the plasmid map of which is shown in FIG. 2a.

Firstly, using the plasmid pET-30a(+)-FS4-SpyCatcher (SEQ ID NO: 9) as a DNA template, primers SpyCatcher-F and SpyCatcher-R in the Table 1 were used for PCR amplification to obtain a polynucleotide fragment Nde I-PT linker-SpyCatcher-Xho I. Q5 high-fidelity DNA polymerase (NEB, M0491) was used in the PCR reaction, and the system for PCR is shown in Table 2.

TABLE 2
PCR Reaction System and Procedure

| Reagent | Amount | |
|---|---|---|
| SpyCatcher-F (10 μM) | 2.5 | μL |
| SpyCatcher-R (10 μM) | 2.5 | μL |
| Template DNA | 1 | ng |
| 5 × Q5 Reaction buffer solution | 10 | μL |
| Q5 DNA polymerase | 0.5 | μL |
| 2.5 mM dNTPs | 4 | μL |

| | |
|---|---|
| ddH₂O | Supplemented to 50 μL |

PCR reaction procedure: a) 98° C. for 30 sec; b) 98° C. for 10 sec; c) 57° C. for 15 sec; d) 72° C. for 15 sec; e) 30 cycles of steps b)-d); f) 72° C. for 2 min; and g) 4° C. hold.

After the completion of the PCR reaction, the PCR amplification product was detected by 1% agarose gel electrophoresis, and the result showed that a target DNA band as expected was obtained by PCR amplification. An ultra-thin DNA product purification kit (Tiangen Biotech (Beijing) Co., Ltd., DP203-02) was used to purify and recover the target DNA which then was preserved at −20° C. for later use.

The purified and recovered target DNA was doubly digested with restriction enzymes Nde I (NEB, RO111L) and Xho I (NEB, R0146L) and then linked to the plasmid pET-30a(+) (Novagen) which was doubly digested with the same enzymes. The linked product was transformed to competent cells of Escherichia coli BL21 (DE3) (Novagen). The transformed cells were spread on a Luria-Bertani medium (LB) plate supplemented with 50 μg/mL of Kanamycin (Kan) and positive clones were selected. The plasmid was extracted and sequenced, and the sequencing result indicated that the cloned pET30a(+)-PT linker-SpyCatcher-His sequence was correct.

### 1.2 Construction of Expression Plasmid of SpyTag Fusion Protein

The expression plasmid pET30a(+)-SpyTag-GS linker-RFP-His (SEQ ID NO: 18) constructed in this example was used to express the fusion protein SpyTag-RFP (SEQ ID NO: 24) of a SpyTag and red fluorescence protein (RFP), and the plasmid map was shown in FIG. 2b. The expression plasmid pET30a(+)-SpyTag-GS linker-GA-His was used to express the fusion protein SpyTag-GA (SEQ ID NO: 25) of a SpyTag and glutaryl-7-amidocephalosporanic acid acylase (GA), and the plasmid map was shown in FIG. 2c.

1) Construction of the plasmid pET30a(+)-SpyTag-Gslinker-RFP-His: firstly, using the plasmid pET30a(+)-Spy-RFP (SEQ ID NO: 10) as a DNA template, the primers RFP-F and RFP-R in the Table 1 were used for PCR amplification to obtain the polynucleotide fragment Nde I-SpyTag-Gslinker-RFP-Hind III. Q5 high-fidelity DNA polymerase was used in the PCR reaction, and the PCR system were shown in Table 3.

TABLE 3
PCR Reaction System

| Reagent | Amount | |
|---|---|---|
| RFP-F (10 μM) | 2.5 | μL |
| RFP-R (10 μM) | 2.5 | μL |
| Template DNA | 1 | ng |
| 5 × Q5 Reaction buffer solution | 10 | μL |
| Q5 DNA polymerase | 0.5 | μL |
| 2.5 mM dNTPs | 4 | μL |

| | |
|---|---|
| ddH₂O | Supplemented to 50 μL |

PCR reaction procedure: a) 98° C. for 30 sec; b) 98° C. for 10 sec; c) 57° C. for 20 sec; d) 72° C. for 30 sec; e) 30 cycles of steps b-d; f) 72° C. for 2 min; and g) 4° C. hold.

After the completion of the reaction, the PCR amplification product was detected by 1% agarose gel electrophoresis, and the result showed that a target DNA band as expected was obtained by PCR amplification. An ultra-thin DNA product purification kit was used to purify and recover the target DNA, which was then preserved at −20° C. for later use.

The purified and recovered target DNA was doubly digested with restriction enzymes Nde I and Hind III (NEB, R3104L) and then linked to the plasmid pET-30a(+) which was doubly digested with the same enzymes. The linked product was transformed to competent cells of E. coli. BL21(DE3). The transformed cells were spread onto an LB plate supplemented with 50 μg/mL of Kan and positive clones were selected. The plasmid was extracted and sequenced, and the sequencing result indicated that the cloned pET30a(+)-SpyTag-Gslinker-RFP-His sequence was correct.

2) Construction of the plasmid pET30a(+)-SpyTag-Gslinker-GA-His (SEQ ID NO: 19): the GA encoding sequence (SEQ ID NO: 16) of Pseudomonas sp. SY-77 was synthesized after codon optimization by Sangon Biotech (Shanghai) Co., Ltd., the protein sequence of GA shown in SEQ ID NO: 26. Primers GA-F and GA-R in the Table 1 were used for PCR amplification to obtain the polynucleotide fragment Sac I-GA-Hind III. Q5 high-fidelity DNA polymerase was used in the PCR reaction, and the PCR system and procedure were shown in Table 4.

TABLE 4
PCR Reaction System

| Reagent | Amount | |
|---|---|---|
| GA-F (10 μM) | 2.5 | μL |
| GA-R (10 μM) | 2.5 | μL |
| Template DNA | 1 | ng |
| 5 × Q5 Reaction buffer solution | 10 | μL |
| High GC reaction buffer solution | 10 | μL |
| Q5 DNA polymerase | 0.5 | μL |
| 2.5 mM dNTPs | 4 | μL |

| | |
|---|---|
| ddH₂O | Supplemented to 50 μL |

PCR reaction procedure: a) 98° C. for 30 sec; b) 98° C. for 10 sec; c) 57° C. for 20 sec; d) 72° C. for 90 sec; e) 30 cycles of steps b-d; f) 72° C. for 4 min; and g) 4° C. hold.

After the completion of the reaction, the PCR amplification product was detected by 1% agarose gel electrophoresis, and the result showed that a target DNA band as expected was obtained by PCR amplification. An ultra-thin DNA product purification kit was used to purify and recover the target DNA which was then preserved at −20° C. for later use.

The purified and recovered target DNA was doubly digested with restriction enzymes Sac I (NEB, R0156L) and Hind III and then linked to the plasmid pET30a(+)-SpyTag-Gslinker-GA-His which was doubly digested with the same enzymes. The linked product was transformed to competent cells of Escherichia coli BL21(DE3). The transformed cells were spread onto an LB plate supplemented with 50 g/mL of Kan and positive clones were selected. The plasmid was extracted and sequenced, and the sequencing result indicated that the cloned pET30a(+)-SpyTag-Gslinker-GA-His sequence was correct.

Such plasmids could be prepared by a person skilled in the art through conventional operations.

### Example 2: Expression, Purification, and Activity Determination of Fusion Proteins

### 2.1 Expression of Fusion Proteins

The strain (containing the plasmid pET30a(+)-PT linker-SpyCatcher-His) constructed in the Example 1 was inoculated to an LB liquid medium containing 50 μg/mL of kanamycin (Kan) in an inoculum amount of 1:50, cultured overnight, then transferred to a fresh LB medium (50 μg/mL of Kan) and cultured to the logarithmic phase (OD₆₀₀=0.4-0.6) in a shaker at 37° C., added with isopropyl-beta-D-thiogalactoside (IPTG) to a final concentration of 0.2 mM, and induced at 30° C. for 6 hours. The cell concentration OD₆₀₀ was measured, and cells were harvested.

The strain (containing the plasmid pET30a(+)-SpyTag-GS linker-RFP-His) constructed in the Example 1 was inoculated to an LB liquid medium containing 50 μg/mL of Kan in an inoculum amount of 1:50. The broth cultured overnight was transferred to a fresh LB medium (50 μg/mL of Kan) and cultured to the logarithmic phase (OD₆₀₀=0.4-0.6) in a shaker at 37° C., added with IPTG to a final concentration of 0.2 mM, and induced at 23° C. for 22 hours. The cell concentration OD₆₀₀ was measured, and cells were harvested.

The strain (containing the plasmid pET30a(+)-SpyTag-GS linker-GA-His) constructed in the Example 1 was inoculated to an LB liquid medium containing 50 μg/mL of Kan in an inoculum amount of 1:50. The broth cultured overnight was transferred to a fresh LB medium (50 μg/mL of Kan) and cultured to the logarithmic phase (OD₆₀₀=0.4-0.6) in a shaker at 37° C., added with IPTG to a final concentration of 0.1 mM, and induced at 18° C. for 12 hours. The cell concentration OD₆₀₀ was measured, and cells were harvested.

### 2.2 SDS-PAGE Detection and Quantitation

Cells from step 2.1 were harvested and resuspended with a PBS buffer (0.1 M, pH 7.0) to 50 OD/mL. The cells were disrupted by ultrasonication on ice (under conditions of: power: 200 W; ultrasonication time: 3 sec; interval time: 3 sec; and ultrasonication times: 99). After the completion of the ultrasonication, the supernatant and the precipitate of the buffer solution were separated by centrifugation. To remove soluble components mixed with the precipitate as much as possible, the resulting precipitate was washed twice with the buffer solution of equivalent volume. The supernatant and the resuspension solution of the precipitate were directly used for SDS-PAGE determination. With BSA at gradient concentrations (31.25 μg/mL, 62.5 g/mL, 125 μg/mL, 250 μg/mL, and 500 μg/mL) as quantitation standards, quantitation was performed by using ImageJ software (National Institutes of Health, USA).

### 2.3 Purification and Dialysis of Fusion Proteins

The cells in the Example 2.1 were harvested and resuspended with a PBS buffer (0.1 M, pH 7.0) to 50 OD/mL. The cells were disrupted by a high pressure homogenizer (PhD Technology International LLC, USA) (under conditions of: pressure: 12,000 psi; and disruption cycles: 3). After the completion of disruption by high pressure homogenization, the supernatant and the precipitate of the lysis solution were separated by centrifugation. The supernatant were collected and purified by nickel column affinity chromatography. Samples before and after the purification and the flow-through were collected, and the effect of purification was detected by SDS-PAGE.

The purified protein supernatant was dialyzed with a PBS (0.1 M, pH 7.0) as a dialysis buffer.

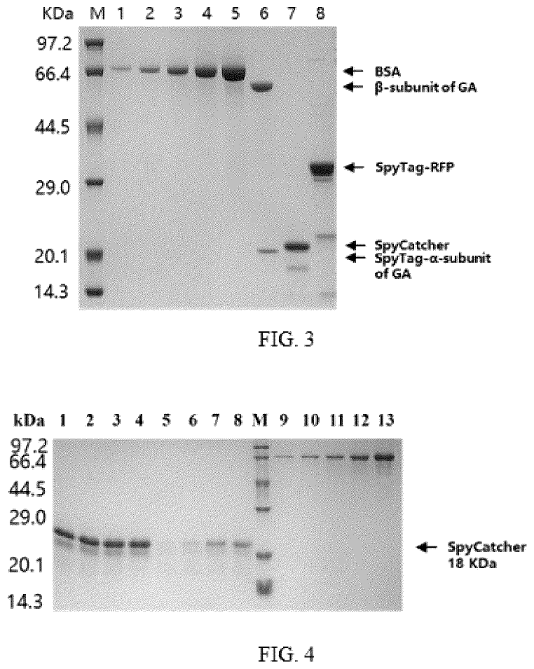

Analysis results of SDS-PAGE were shown in FIG. 3. Lanes 6, 7 and 8 represented fusion proteins SpyTag-GSlinker-GA (hereinafter referred to as SpyTag-GA for the sake of simplicity), PT linker-SpyCatcher (hereinafter referred to as SpyCatcher for the sake of simplicity) and SpyTag-Gslinker-RFP (hereinafter referred to as SpyTag-RFP for the sake of simplicity), respectively, and the three purified fusion proteins had the purities of 95%, 81%, and 88%, respectively, and could be used for subsequent immobilization experiments.

### 2.4 Enzyme Activity and Fluorescence Determination of Fusion Proteins

Cells from the step 2.1 were harvested and resuspended with a PBS buffer (0.1 M, pH 7.0) to 50 OD/mL. The cells were disrupted by a high pressure homogenizer (PhD Technology International LLC, USA) (under conditions of: pressure: 12,000 psi; and disruption cycles: 3). After the completion of disruption by high pressure homogenization, the supernatant and the precipitate of the lysis solution were separated by centrifugation. The supernatant was directly used for respective enzyme activity and fluorescence determination. Methods for determining the enzyme activity of GA and the fluorescence of RFP were as follows:

Determination principle of the GA enzyme activity: glutaryl-7-aminocephalosporanic acid (Gl-7-ACA) was hydrolyzed into 7-aminocephalosporanic acid (7-ACA) under the catalysis of GA. A primary amino group of 7-ACA reacted with p-dimethylaminobenzaldehyde (pDAB) to generate yellow Schiffs base having the maximum absorbance at 415 nm.

Determination of the free GA enzyme activity: 20 μL of enzyme solution (in a 0.1M PBS at pH 7.0) was mixed with 20 μL of the substrate (1% (w/v) GL-7-ACA in a 0.1M PBS at pH 7.0), and incubated at 37° C. for 10 minutes. 140 μL of mixture of 20% (v/v) acetic acid and 0.05M NaOH (2:1, v/v) was added thereto to terminate the reaction, and then added with 20 μL of 0.5% (w/v) pDAB. The mixture was continuously incubated at 25° C. for 10 minutes, and then its absorbance at 415 nm was measured. One GA activity unit (U) was defined as an amount of enzyme needed to generate 1 μmol of 7-ACA per minute at 37° C. and pH 7.0.

The enzyme activity of free GA was calculated by:

$$free enzyme activity = \frac{ΔA / min \times Vt \times X}{S \times d \times Vs} U / mL .$$

wherein,
ΔA/min represents a variation value of absorbance per minute, namely a slope;
S represents molar extinction coefficient of 7-ACA (1.576 cm²/μmoL), obtained from a standard curve;
d represents optical length (if the reaction system is 0.2 mL, the optical length is about 0.5 cm);
Vt represents total volume of the reaction mixture (0.2 mL);
Vs represents the volume of a sample enzyme solution (0.02 mL); and
X represents the dilution factor of a sample enzyme solution.

Fluorescence detection of free RFP: the purified RFP was diluted appropriately, and then 200 μL of the diluted RFP was added to a black flat-bottomed 96-well plate (Corning, 3925). The fluorescence intensity of RFP was detected by using a raster type multifunctional microplate reader (infinite M200 PRO, TECAN) at 37° C., with an excitation wavelength of 588 nm, an emission wavelength of 635 nm, and a gain value of 100% [Anuar I N A K, Banerjee A, Keeble A H, et al. Spy&Go purification of SpyTag-proteins using pseudo-SpyCatcher to access an oligomerization toolbox[J]. Nature communications, 2019, 10(1): 1734.].

### Example 3: Preparation of SpyCatcher Modified Carrier

In the present invention, a SpyCatcher modified carrier was obtained firstly and then used to immobilize a SpyTag fusion protein by the Spy chemistry through one step.

### 3.1 Preparation of SpyCatcher Modified Carrier

50 mg of epoxy carrier LX-1000EP (Xian Sunresin New Materials Co. Ltd.) was weighed. The LX-1000EP was washed with PBS (0.1 M, pH 7.0) for 3 times, and added with 0.25, 0.5, 1, or 2 mg of purified SpyCatcher to react on a miniature rotator (Gilson, Roto-Mini Plus) at 25° C. and a speed of 25 rpm for 12 hours.

Meanwhile, an equal amount of SpyCatcher supernatant was used as a blank control, and its protein content was detected.

After the completion of the reaction, the supernatant was removed by centrifugation, and washed with PBS (0.1 M, pH 7.0) for 5 times, then the SpyCatcher modified carrier (hereinafter referred to as SpyCatcher-LX-1000EP for the sake of simplicity) was obtained. The immobilization amount and the immobilization efficiency of SpyCatcher were calculated by the following formulas:

SpyCatcher immobilization amount (mg)=the total protein amount (mg) in the supernatant before immobilization−the total protein amount (mg) in the supernatant after immobilization

$$SpyCatcher immobilization efficiency ( \% ) = \frac{SpyCatcher immobilization amount ( mg )}{\begin{matrix}total protein amount ( mg ) in SpyCatcher supernatant before \\ immobilization\end{matrix}} \times 100 \%$$

The analysis results of SDS-PAGE were shown in FIG. 4. In accordance with the protein quantitation standard, optical density analysis was performed on target bands by ImageJ gel quantitation analysis software, and the immobilization efficiency (Table 5) of the fusion protein SpyCatcher was calculated. From the table, as the added SpyCatcher increased, the immobilization efficiency of SpyCatcher decreased accordingly. When 20 mg/g of carrier was added, the immobilization efficiency could still be kept at 87.5%, which did not decrease too much as compared with addition of 10 mg/g of carrier, whereas when the amount added was increased to 40 mg/g of carrier, the immobilization efficiency was reduced to 63.7%. Thus, the amount of SpyCatcher added was determined as 20 mg/g of carrier in other experiments in the present invention.

TABLE 5
Immobilization Results of SpyCatcher

| Amount of Protein | Immobilization Amount of | Immobilization |
|---|---|---|
| added (mg/g of carrier) | SpyCatcher (mg/g of carrier) | Efficiency (%) |
| 5 | 5 | 100.0 |
| 10 | 9.5 | 95.0 |
| 20 | 17.5 | 87.5 |
| 40 | 25.5 | 63.7 |

The resultant immobilization SpyCatcher-LX-1000EP was added with a blocking buffer solution (3M glycine, pH 8.5) in a volume of 12 times to react on a miniature rotator (Gilson, Roto-Mini Plus) at 25° C. and a speed of 25 rpm for 24 hours. After the completion of blocking, the blocking solution was removed by centrifugation, and the left was washed with PBS (0.1 M, pH 7.0) for 3 times and preserved at 4° C.

### Example 4: Immobilization of Fusion Proteins

### 4.1 Immobilization of SpyTag-RFP at Different pHs

The purified SpyTag-RFP was immobilized with the SpyCatcher-LX-1000EP at different pHs.

The present method: 50 mg of SpyCatcher-LX-1000EP was weighed, washed with 0.1M PBS (respectively at pH 5.0, 6.0, 7.0, and 8.0) for 3 times, and added with 0.75 mg of purified SpyTag-RFP (3.75 mg/mL) (respectively at pH 5.0, 6.0, 7.0, and 8.0) to react on a miniature rotator (Gilson, Roto-Mini Plus) at 25° C. and a speed of 25 rpm for 2 hours.

Blocking control: 50 mg of an epoxy carrier after a blocking reaction was weighed, washed with 0.1M PBS (respectively at pH 5.0, 6.0, 7.0, and 8.0) for 3 times, and added with 0.75 mg of the purified SpyTag-RFP (3.75 mg/mL) (respectively at pH 5.0, 6.0, 7.0, and 8.0) to react on a miniature rotator (Gilson, Roto-Mini Plus) at 25° C. and a speed of 25 rpm for 2 hours. This was used as a blocking control group.

Epoxy method: 50 mg of LX-1000EP was weighed, washed with 0.1M PBS (respectively at pH 5.0, 6.0, 7.0, and 8.0) for 3 times, and added with 0.75 mg of the purified SpyTag-RFP (3.75 mg/mL) (respectively at pH 5.0, 6.0, 7.0, and 8.0) to react on a miniature rotator (Gilson, Roto-Mini Plus) at 25° C. and a speed of 25 rpm for 2 hours. This was used as a control group.

Meanwhile, an equal amount of SpyTag-RFP supernatant was used as a blank control, and its protein content was detected.

After the completion of the reaction, the immobilized RFP was isolated by centrifugation. The immobilized RFP was obtained after washing with PBS (0.1 M, pH 7.0) for 5 times. The immobilization amount and the immobilization efficiency of the immobilized RFP were calculated by the following formulas:

$$RFP immobilization amount ( mg ) = the total protein amount ( mg ) in the supernatant before immobilization - the total protein amount ( mg ) in the supernatant after immobilization - the total protein amount ( mg ) in the washing liquid supernatant$$

$$RFP immobilization efficiency ( \% ) = \frac{RFP immobilization amount ( mg )}{\begin{matrix}total protein amount ( mg ) in RFP supernatant before \\ immobilization\end{matrix}} \times 100 \%$$

The results of immobilization were shown in Table 6. The immobilization of the protein in a range of pH 5-8: the epoxy method exhibited increased immobilization efficiency and increased recovery rate of activity with increasing pH, the immobilization efficiencies being 31-41% and the recovery rates of activity being 22-43%. The present method exhibited the highest immobilization efficiency and recovery rate of activity at pH 6-7, the immobilization efficiencies being 51-62% and the recovery rates of activity being 51-67%.

TABLE 6
Results of Immobilization of SpyTag-RFP with
SpyCatcher-LX-1000EP at different pHs

| | | Amount of | Immobiliza- | Recovery |
|---|---|---|---|---|
| | | Protein added | tion | Rate of |
| | Immobilization | (mg/g of | efficiency | Activity |
| pH | Method | carrier) | (%) | (%) |
| 5 | The present method | 15 | 57.1 | 58.9 |
| | Blocking control | 15 | 19.9 | 6.6 |
| | Epoxy method | 15 | 30.9 | 22.1 |
| 6 | The present method | 15 | 58.9 | 66.9 |
| | Blocking control | 15 | 33.7 | 3.5 |
| | Epoxy method | 15 | 35.9 | 30.5 |
| 7 | The present method | 15 | 62.1 | 67.1 |
| | Blocking control | 15 | 34.4 | 3.0 |
| | Epoxy method | 15 | 36.9 | 33.8 |
| 8 | The present method | 15 | 51.1 | 51.2 |
| | Blocking control | 15 | 36.1 | 4.6 |
| | Epoxy method | 15 | 41.4 | 43.0 |

### 4.2 Immobilization of SpyTag-RFP at Different Temperatures

The purified SpyTag-RFP was immobilized with the SpyCatcher-LX-1000EP at different temperatures.

The present method: 50 mg of SpyCatcher-LX-1000EP was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 4° C., 25° C., and 37° C., respectively, and a speed of 25 rpm for 2 hours.

Blocking control: 50 mg of an epoxy carrier after a blocking reaction was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 4° C., 25° C., and 37° C., respectively, and a speed of 25 rpm for 2 hours. This was used as a blocking control group.

Epoxy method: 50 mg of LX-1000EP was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 4° C., 25° C., and 37° C., respectively, and a speed of 25 rpm for 2 hours. This was used as a control group.

Meanwhile, an equal amount of SpyTag-RFP supernatant was used as a blank control, and its protein content was detected.

After the completion of the reaction, the immobilized RFP was isolated by centrifugation. The immobilized RFP was obtained after being washed with PBS (0.1 M, pH 7.0) for 5 times. The immobilization amount and the immobilization efficiency of the immobilized RFP were calculated by the following formulas:

$$RFP immobilization amount ( mg ) = the total protein amount ( mg ) in the supernatant before immobilization - the total protein amount ( mg ) in the supernatant after immobilization - the total protein amount ( mg ) in the washing liquid supernatant$$

$$RFP immobilization efficiency ( \% ) = \frac{RFP immobilization amount ( mg )}{\begin{matrix}total protein amount ( mg ) in RFP supernatant before \\ immobilization\end{matrix}} \times 100 \%$$

The results of immobilization were shown in Table 7. The immobilization of the protein at temperatures 4° C., 25° C., and 37° C.: the epoxy method exhibited increased immobilization efficiency and increased recovery rate of activity along with increasing temperature, the immobilization efficiencies being 26-40% and the recovery rates of activity being 34-47%. The present method was insensitive to the temperature, and exhibited the immobilization efficiency of 62% and the recovery rates of activity of 65-67% at 4° C., 25° C., and 37° C.

TABLE 7
Immobilization of SpyTag-RFP with SpyCatcher-
LX-1000EP at Different Temperatures

| | | Amount of | | |
|---|---|---|---|---|
| | | Protein | Immobiliza- | Recovery |
| Temper- | | added | tion | Rate of |
| ature | Immobilization | (mg/g of | efficiency | Activity |
| (° C.) | Method | carrier) | (%) | (%) |
| 4 | The present method | 15 | 62.2 | 67.2 |
| | Blocking control | 15 | 23.2 | 3.8 |
| | Epoxy method | 15 | 26.1 | 33.8 |
| 25 | The present method | 15 | 62.1 | 67.1 |
| | Blocking control | 15 | 34.4 | 3.0 |
| | Epoxy method | 15 | 36.9 | 33.8 |
| 37 | The present method | 15 | 62.3 | 64.7 |
| | Blocking control | 15 | 22.1 | 14.4 |
| | Epoxy method | 15 | 40.5 | 47.4 |

### 4.3 Immobilization of SpyTag-RFP/SpyTag-GA

### 4.3.1 Immobilization of SpyTag-RFP

The present method: 50 mg of SpyCatcher-LX-1000EP was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 25° C. and a speed of 25 rpm for 2 hours.

Epoxy method: 50 mg of LX-1000EP was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 25° C. and a speed of 25 rpm for 2 hours. This was used as a control group.

Blocking control: 50 mg of an epoxy carrier after a blocking reaction was weighed, washed with PBS (0.1 M, pH 7.0) for 3 times, and added with 0.75 mg of the purified SpyTag-RFP (3.75 mg/mL) to react on a miniature rotator (Gilson, Roto-Mini Plus) at 25° C. and a speed of 25 rpm for 2 hours. This was used as a blocking control group.

Meanwhile, an equal amount of SpyTag-RFP supernatant was used as a blank control, and its protein content was detected.

After the completion of the reaction, the immobilized RFP was isolated by centrifugation. The immobilized RFP was obtained after being washed with PBS (0.1 M, pH 7.0) for 5 times. The immobilization amount and the immobilization efficiency of the immobilized RFP were calculated by the following formulas:

RFP immobilization amount (mg)=the total protein amount (mg) in the supernatant before immobilization−the total protein amount (mg) in the supernatant after immobilization−the total protein amount (mg) in the washing liquid supernatant

$$RFP immobilization efficiency ( \% ) = \frac{RFP immobilization amount ( mg )}{\begin{matrix}total protein amount ( mg ) in RFP supernatant before \\ immobilization\end{matrix}} \times 100 \%$$

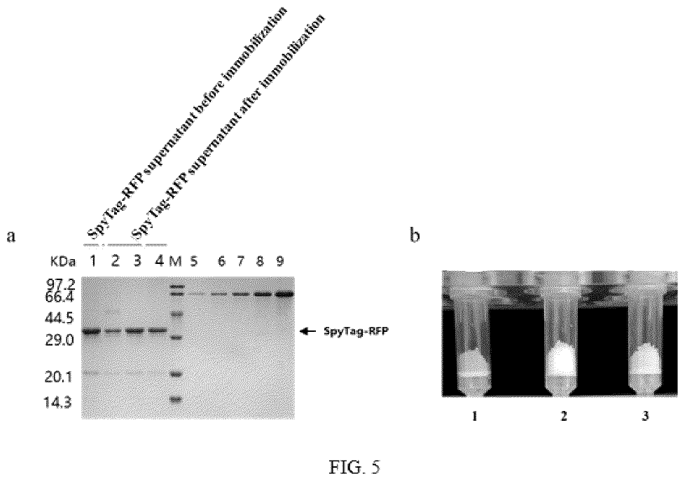

The analysis results of SDS-PAGE were shown in FIG. 5a. In accordance with the protein quantitation standard, optical density analysis was performed on target bands by ImageJ gel quantitative analysis software, and the immobilization efficiency of the fusion protein SpyTag-RFP could be calculated, the results as shown in Table 8. The blocking control exhibited the immobilization efficiency of SpyTag-RFP immobilized of 13.4%, while the epoxy immobilization method exhibited the immobilization efficiency of 29.6%, and the present method exhibited the immobilization efficiency of 66.0%. Such fluorescences could also be directly observed from the immobilization carriers, the results shown in FIG. 5b.

TABLE 8
Immobilization Results of SpyTag-RFP

| | Amount of Fusion | Immobilization |
|---|---|---|
| | Protein added | Efficiency |
| Immobilization Method | (mg/g of carrier) | (%) |
| Blocking control | 15 | 13.4 |
| Epoxy immobilization method | 15 | 29.6 |
| The present method | 15 | 66.0 |

4.3.2 Direct Immobilization of SpyTag-RFP from Cell Lysis Solution

The present method: 50 mg of SpyCatcher-LX-1000EP was weighed, washed with PBS (0.1 M, pH 7.0) for 3 times, and added with 0.2 mL of the supernatant of the lysate of Escherichia coli cells overexpressing SpyTag-RFP to react on a miniature rotator (Gilson, Roto-Mini Plus) at 25° C. and a speed of 25 rpm for 2 hours.

Epoxy method: 50 mg of LX-1000EP was weighed, washed with PBS (0.1 M, pH 7.0) for 3 times, and added with 0.2 mL of the supernatant of the lysate of Escherichia coli cells overexpressing SpyTag-RFP to react on a miniature rotator (Gilson, Roto-Mini Plus) at 25° C. and a speed of 25 rpm for 2 hours. This was used as a control group.

Blocking control: 50 mg of an epoxy carrier after a blocking reaction was weighed, washed with PBS (0.1 M, pH 7.0) for 3 times, and added with 0.2 mL of the supernatant of the lysate of Escherichia coli cells overexpressing SpyTag-RFP to react on a miniature rotator (Gilson, Roto-Mini Plus) at 25° C. and a speed of 25 rpm for 2 hours. This was used as a blocking control group.

Meanwhile, an equal amount of SpyTag-RFP supernatant was used as a blank control, and its protein content was detected.

After the completion of the reaction, the immobilized RFP was isolated by centrifugation. The immobilized RFP isolated was washed with PBS (0.1 M, pH 7.0) for 5 times. The immobilized SpyTag-RFP was obtained. The immobilization amount, the immobilization efficiency and the recovery rate of fluorescence of the immobilized RFP were calculated by the following formulas:

$$RFP immobilization amount ( mg ) = the total protein amount ( mg ) in the supernatant before immobilization - the total protein amount ( mg ) in the supernatant after immobilization - the total protein amount ( mg ) in the washing liquid supernatant$$

$$RFP immobilization efficiency ( \% ) = \frac{RFP immobilization amount ( mg )}{\begin{matrix}total protein amount ( mg ) in RFP supernatant before \\ immobilization\end{matrix}} \times 100 \%$$

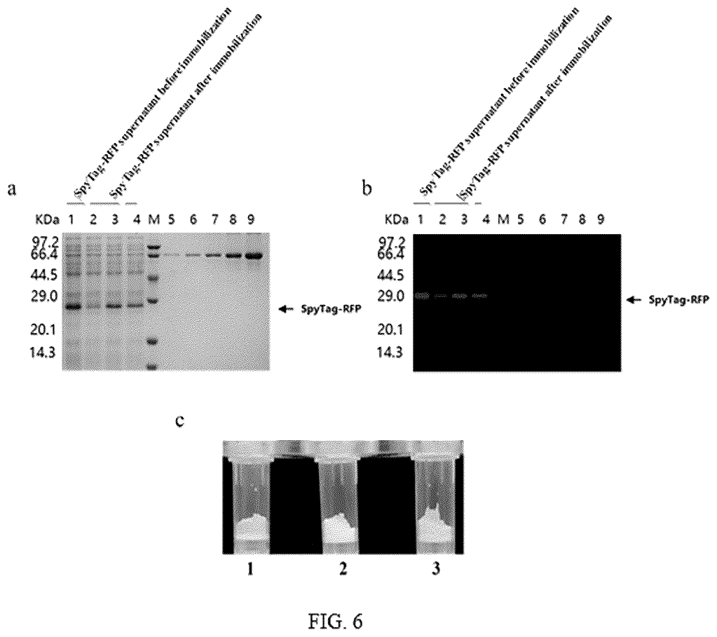

The analysis results of SDS-PAGE and fluorescence PAGE were shown in FIG. 6a and FIG. 6b. In accordance with the protein quantitation standard, optical density analysis was performed on target bands by ImageJ gel quantitative analysis software, and the immobilization efficiency of the fusion protein SpyTag-RFP in the cell lysis solution could be calculated, with the results being as shown in Table 9. The blocking control exhibited the immobilization efficiency of SpyTag-RFP of 42.3%, while the epoxy immobilization method exhibited the immobilization efficiency of 62.7%, and the present method exhibited the immobilization efficiency of being increased to 90.8%. The immobilization results of SpyTag-RFP could also be directly observed through colors on the immobilization carriers (FIG. 6c).

TABLE 9
Results of Immobilization of SpyTag-RFP from Cell Lysis Solution

| | Amount of Fusion | Immobilization |
|---|---|---|
| | Protein Added^{a} | Efficiency |
| Immobilization Method | (mg/g of carrier) | (%) |
| Blocking control | 5.8 | 42.3 |
| Epoxy immobilization method | 5.8 | 62.7 |
| The present method | 5.8 | 90.8 |

^{a}The amount of the fusion protein added was estimated by SDS-PAGE.

### 4.3.3 Immobilization of SpyTag-GA

The present method: 50 mg of SpyCatcher-LX-1000EP was weighed, washed with PBS (0.1 M, pH 7.0) for 3 times, and added with 0.75 mg of the purified SpyTag-GA (3.75 mg/mL) to react on a miniature rotator (Gilson, Roto-Mini Plus) at 25° C. and a speed of 25 rpm for 2 hours.

Epoxy method: 50 mg of LX-1000EP was weighed, washed with PBS (0.1 M, pH 7.0) for 3 times, and added with 0.75 mg of the purified SpyTag-GA (3.75 mg/mL) to react on a miniature rotator (Gilson, Roto-Mini Plus) at 25° C. and a speed of 25 rpm for 2 hours. This was used as a control group.

Blocking control: 50 mg of an epoxy carrier after a blocking reaction was weighed, washed with PBS (0.1 M, pH 7.0) for 3 times, and added with 0.75 mg of the purified SpyTag-GA (3.75 mg/mL) to react on a miniature rotator (Gilson, Roto-Mini Plus) at 25° C. and a speed of 25 rpm for 2 hours. This was used as a blocking control group.

Meanwhile, an equal amount of SpyTag-GA supernatant was used as a blank control, and its protein content was detected.

After the completion of the reaction, the immobilized enzyme GA was isolated by centrifugation. The immobilized enzyme GA isolated was washed with PBS (0.1 M, pH 7.0) for 5 times. The immobilized GA was obtained. The immobilization amount, the immobilization efficiency and the recovery rate of enzyme activity of the immobilized enzyme GA were calculated by the following formulas:

$$GA immobilization amount ( mg ) = the total protein amount ( mg ) in the supernatant before immobilization - the total protein amount ( mg ) in the supernatant after immobilization - the total protein amount ( mg ) in the washing liquid supernatant$$

$$GA immobilization efficiency ( \% ) = \frac{GA immobilization amount ( mg )}{\begin{matrix}total protein amount ( mg ) in GA supernatant before \\ immobilization\end{matrix}} \times 100 \%$$

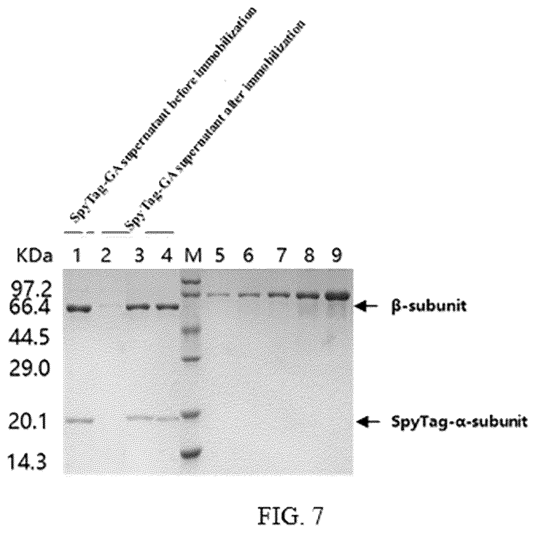

The analysis results of SDS-PAGE were shown in FIG. 7. In accordance with the protein quantitation standard, optical density analysis was performed on target bands by ImageJ gel quantitative analysis software, and the immobilization efficiency of the fusion protein SpyTag-GA could be calculated, with the results being as shown in Table 10. The blocking control exhibited the immobilization efficiency of SpyTag-GA of 28.4%, while the epoxy immobilization method exhibited the immobilization efficiency of 29.4% and the present method exhibited the immobilization efficiency of being increased to 91.5%.

TABLE 10
Immobilization Results of SpyTag-GA

| | Amount of | Immobilization |
|---|---|---|
| | Protein added | Efficiency |
| Immobilization Method | (mg/g of carrier) | (%) |
| Blocking control | 15 | 28.4 |
| Epoxy immobilization method | 15 | 29.4 |
| The present method | 15 | 91.5 |

4.3.4 Direct Immobilization of SpyTag-GA from Cell Lysis Solution

50 mg of SpyCatcher-LX-1000EP was weighed, washed with PBS (0.1 M, pH 7.0) for 3 times, and added with 0.2 mL of the supernatant of the lysate of Escherichia coli cells overexpressing SpyTag-GA to react on a miniature rotator (Gilson, Roto-Mini Plus) at 25° C. and a speed of 25 rpm for 2 hours.

50 mg of LX-1000EP was weighed, washed with PBS (0.1 M, pH 7.0) for 3 times, and added with 0.2 mL of the supernatant of the lysate of Escherichia co/i cells overexpressing SpyTag-GA to react on a miniature rotator (Gilson, Roto-Mini Plus) at 25° C. and a speed of 25 rpm for 2 hours. This was used as a control group.

50 mg of an epoxy carrier after a blocking reaction was weighed, washed with PBS (0.1 M, pH 7.0) for 3 times, and added with 0.2 mL of the supernatant of the lysate of Escherichia co/i cells overexpressing SpyTag-GA to react on a miniature rotator (Gilson, Roto-Mini Plus) at 25° C. and a speed of 25 rpm for 2 hours. This was used as a blocking control group.

Meanwhile, an equal amount of SpyTag-GA supernatant was used as a blank control, and its protein content was detected.

After the completion of the reaction, the immobilized enzyme GA was isolated by centrifugation. The immobilized enzyme GA isolated was washed with PBS (0.1 M, pH 7.0) for 5 times. The immobilized SpyTag-GA was obtained. The immobilization amount, the immobilization efficiency and the recovery rate of activity of SpyTag-GA were calculated by the following formulas:

$$GA immobilization amount ( mg ) = the total protein amount ( mg ) in the supernatant before immobilization - the total protein amount ( mg ) in the supernatant after immobilization - the total protein amount ( mg ) in the washing liquid supernatant$$

$$GA immobilization efficiency ( \% ) = \frac{GA immobilization amount ( mg )}{\begin{matrix}total protein amount ( mg ) in GA supernatant before \\ immobilization\end{matrix}} \times 100 \%$$

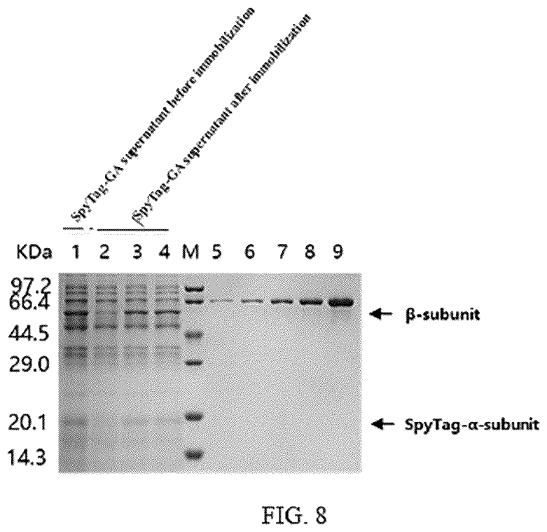

The results of SDS-PAGE were shown in FIG. 8.

In accordance with the protein quantitation standard, optical density analysis was performed on target bands by ImageJ gel quantitative analysis software, and the immobilization efficiency of the fusion protein SpyTag-GA in the cell lysis solution could be calculated, with the results being as shown in Table 11. The blocking control exhibited the immobilization efficiency of SpyTag-GA of 28.5%, while the epoxy immobilization method exhibited the immobilization efficiency of 25.0% and the present method exhibited the immobilization efficiency of being increased to 86.2%.

TABLE 11
Results of Immobilization of SpyTag-GA from Cell Lysis Solution

| | Amount of | Immobilization |
|---|---|---|
| | Protein added ^{a} | Efficiency |
| Immobilization Method | (mg/g of carrier) | (%) |
| Blocking control | 2.0 | 28.5 |
| Epoxy immobilization method | 2.0 | 25.0 |
| The present method | 2.0 | 86.2 |

^{a} The amount of the fusion protein added was estimated by SDS-PAGE.

### 4.4 Activity Detection of Immobilized SpyTag-RFP/GA

Fluorescence detection of immobilized SpyTag-RFP: 2 mg (appropriate amount) of immobilized RFP was weighed and added to a black flat-bottomed 96-well plate. Meanwhile, 0.2 mL of PBS (0.1M, pH 7.0) was added thereto for sufficient resuspension. The fluorescence intensity of the immobilized SpyTag-RFP was detected by using a raster type multifunctional microplate reader (TECAN, infinite M200 PRO) at 37° C., with an excitation wavelength of 588 nm, an emission wavelength of 635 nm, and a gain value of 100%.

Determination of the enzyme activity of immobilized SpyTag-GA: 4 mg of immobilized SpyTag-GA was weighed, added with 0.5 mL of 0.1M PBS (pH 7.0), mixed with 0.5 mL of the substrate (1% (w/v) GL-7-ACA in 0.1M PBS at pH 7.0), and incubated at 37° C. for 10 minutes. Next, 0.2 mL of the reaction solution was taken, firstly added with 0.7 mL of the mixture of 200% (v/v) acetic acid and 0.05M NaOH (2:1, v/v), and then added with 0.1 mL of 0.5% (w/v) pDAB. The mixture was further incubated at 25° C. for 10 minutes, and then its absorbance at 415 nm was measured.

The enzyme activity of the immobilized SpyTag-GA was calculated by:

$$immobilized enzyme activity = \frac{Δ A / min \times Vt}{S \times d \times M} ( U / g ) ,$$

wherein,
ΔA/min represents a variation value of absorbance per minute, namely a slope;
S represents the molar extinction coefficient of 7-ACA (1.576 cm²/μmoL), calculated from a standard curve;
d represents optical length (if the reaction system is 0.2 mL, the optical length is about 0.5 cm);
Vt represents the total volume of the reaction mixture (1 mL); and
M represents the weighed amount of the immobilized enzyme (0.004 g).

The results of activity detection of the immobilized SpyTag-RFP/SpyTag-GA were shown in Table 12. The blocking control exhibited the recovery rates of activity of 2.8% and 1.1% for the immobilized SpyTag-RFP and SpyTag-GA, respectively, while the epoxy method exhibited the recovery rates of activity of 19.6% and 2.7%, respectively, and the present method exhibited the recovery rates of activity of being increased to 49.4% and 86.4%, respectively. Moreover, the immobilization method could allow direct immobilization of SpyTag-RFP/SpyTag-GA from a cell lysis solution, and the epoxy method exhibited the recovery rates of activity of 36.3% and 3.4%, respectively, while the present method exhibited the recovery rates of activity increased to 85.0% and 91.2%, respectively.

TABLE 12
Results of Activity Detection of SpyTag-RFP/SpyTag-GA

| | Fluorescence Recovery | | Enzyme Activity Recovery | |
|---|---|---|---|---|
| | Rate of RFP ª (%) | | Rate of GA^{b} (%) | |
| | | Cell Lysis | | Cell Lysis |
| Immobilization | Purification | Solution | Purification | Solution |
| Method | SpyTag-RFP | Spy Tag-RFP | SpyTag-GA | Spy Tag-GA |
| Blocking control | 2.8 | 4.3 | 1.1 | 1.6 |
| Epoxy | | | | |
| immobilization | 19.6 | 36.3 | 2.7 | 3.4 |
| method | | | | |
| The present | 49.4 | 85.0 | 86.4 | 91.2 |
| method | | | | |

${a}^{} Fluorescence recovery rate of immobilized RFP ( \% ) = \\ \frac{total fluorescenc e value ( a . u . / g resin ) of immobilized RFP}{\begin{matrix}total fluorescenc e value ( a . u . / g resin ) \\ in RFP supernatant before immobilization\end{matrix}} \times 100$ ${b}^{} Enzyme activity recovery rate of immobilized GA ( \% ) = \frac{total enzyme activity ( U / g resin ) of immobilized enzyme GA}{\begin{matrix}total enzyme activity ( U / g resin ) \\ in GA supernatant before immobilization\end{matrix}} \times 100 \%$

### Example 5: Comparison of Immobilization Effects of Different Epoxy Carriers

5.1 Respective Immobilization of SpyCatcher with Different Epoxy Carriers

Six different epoxy carriers LX-1000EP (Xian Sunresin New Materials Co. Ltd.), LX-107S (Xian Sunresin New Materials Co. Ltd.), LX-103B (Xian Sunresin New Materials Co. Ltd.), HFA001 (Xian Sunresin New Materials Co. Ltd.), ECR8024 (Purolite Corporation, the United States), and ECR8285 (Purolite Corporation, the United States) were modified by SpyCatcher. Reaction conditions and the calculation of the immobilization efficiency were the same as those in Example 3. The results were shown in Table 13.

Information of the 6 resins:

| | | | | | Epoxy Group | |
|---|---|---|---|---|---|---|
| | Functional | Particle | Mechanical | | Content | |
| Product | Group | Size (μm) | Strength | Hydrophilicity | (μmol/g wet) | Material |
| LX-1000EP | Epoxy group | 150-350 | +++ | ++ | 80-100 | Polyacrylate |
| LX-103B | Epoxy group | 100-300 | ++++ | +++++ | 70-90 | |
| LX-107S | Epoxy group | 100-300 | ++++ | +++ | 75-95 | |
| HFA001 | Epoxy group + | 100-300 | ++++ | ++++ | 50-80 | |
| | amino group | | | | | |
| ECR8204 | Epoxy group | 150-300 | +++ | +++ | 585-780 | Polymethylacrylate |
| ECR8285 | Epoxy group | 250-1000 | + | + | 389-518 | |

TABLE 13
SpyCatcher Immobilization Efficiencies
of Different Epoxy Carriers

| | Amount of Protein Added | Immobilization |
|---|---|---|
| Epoxy Carrier | (mg/g of resin) | Efficiency (%) |
| LX-1000EP | 20 | 91.1 |
| LX-107S | 20 | 100.0 |
| LX-103B | 20 | 97.1 |
| HFA001 | 20 | 93.1 |
| ECR8204 | 20 | 100.0 |
| ECR8285 | 20 | 100.0 |

5.2: Immobilization and Purification of SpyTag-RFP with Different Epoxy Carriers Modified by SpyCatcher

Different SpyCatcher modified epoxy carriers were used to immobilize SpyTag-RFP. Immobilization conditions and the calculations of the immobilization efficiency and the recovery rate of fluorescence were the same as those in Example 4.3, and the results were shown in Table 14.

TABLE 14
Results of Immobilization and Purification of SpyTag-RFP
with Different Epoxy Carriers Modified by SpyCatcher

| | | Amount of | | Recovery |
|---|---|---|---|---|
| SpyCatcher | | Protein | Immobiliza- | Rate of |
| Modified | | Added | tion | Fluores- |
| Epoxy | Immobilization | (mg/g of | Efficiency | cence |
| Carrier | Method | carrier) | (%) | (%) |
| LX-1000EP | The present method | 15 | 65.9 | 45.2 |
| | Blocking control | 15 | 24.5 | 1.4 |
| | Epoxy | 15 | 46.4 | 20.5 |
| | immobilization | | | |
| | method | | | |
| LX-107S | The present method | 15 | 100.0 | 67.3 |
| | Blocking control | 15 | 100.0 | 68.8 |
| | Epoxy | 15 | 100.0 | 90.6 |
| | immobilization | | | |
| | method | | | |
| LX-103B | The present method | 15 | 60.7 | 50.2 |
| | Blocking control | 15 | 28.5 | 0.5 |
| | Epoxy | 15 | 55.5 | 25.4 |
| | immobilization | | | |
| | method | | | |
| HFA001 | The present method | 15 | 50.5 | 33.9 |
| | Blocking control | 15 | 5.9 | 0.3 |
| | Epoxy | 15 | 22.0 | 0.3 |
| | immobilization | | | |
| | method | | | |
| ECR8204 | The present method | 15 | 51.6 | 59.4 |
| | Blocking control | 15 | 61.8 | 59.6 |
| | Epoxy | 15 | 95.7 | 66.8 |
| | immobilization | | | |
| | method | | | |
| ECR8285 | The present method | 15 | 100.0 | 93.3 |
| | Blocking control | 15 | 100.0 | 95.7 |
| | Epoxy | 15 | 100.0 | 95.8 |
| | immobilization | | | |
| | method | | | |

5.3 Immobilization of SpyTag-GA from Cell Lysis Solution with SpyCatcher Modified Epoxy Carriers

Different SpyCatcher modified epoxy carriers were used to immobilize SpyTag-GA from a cell lysis solution. Immobilization conditions and the calculations of the immobilization efficiency and the recovery rate of fluorescence were the same as those in Example 4.3, and the results were shown in Table 15.

TABLE 15
Results of Immobilization of SpyTag-GA from Cell Lysis
Solution with SpyCatcher Modified Epoxy Carriers

| | | Amount of | | |
|---|---|---|---|---|
| SpyCatcher | | Protein | Immobiliza | Recovery |
| Modified | | Added | tion | Rate of |
| Epoxy | Immobilization | (mg/g of | Efficiency | Activity |
| Carrier | Method | carrier) | (%) | (%) |
| LX-1000EP | The present method | 10 | 77.6 | 80.6 |
| | Blocking control | 10 | 33.6 | 2.3 |
| | Epoxy method | 10 | 31.9 | 3.5 |
| LX-107S | The present method | 10 | 49.1 | 55.0 |
| | Blocking control | 10 | 72.5 | 47.1 |
| | Epoxy method | 10 | 58.6 | 69.6 |
| LX-103B | The present method | 10 | 77.0 | 76.0 |
| | Blocking control | 10 | 47.3 | 1.7 |
| | Epoxy method | 10 | 37.3 | 2.6 |
| HFA001 | The present method | 10 | 79.4 | 81.1 |
| | Blocking control | 10 | 46.2 | 13.0 |
| | Epoxy method | 10 | 27.8 | 8.1 |
| ECR8204 | The present method | 10 | 52.1 | 19.1 |
| | Blocking control | 10 | 31.6 | 6.2 |
| | Epoxy method | 10 | 29.1 | 12.0 |
| ECR8285 | The present method | 10 | 57.4 | 28.8 |
| | Blocking control | 10 | 49.6 | 21.8 |
| | Epoxy method | 10 | 59.1 | 30.2 |

5.4 Direct Immobilization of SpyTag-RFP from Cell Lysis Solution with Different Epoxy Carriers Modified by SpyCatcher

Different SpyCatcher modified epoxy carriers were used to immobilize SpyTag-RFP from a cell lysis solution. Immobilization conditions and the calculations of the immobilization efficiency and the recovery rate of fluorescence were the same as those in Example 4.3, and the results were shown in Table 16.

TABLE 16
Results of Immobilization of SpyTag-RFP from Cell Lysis
Solution with SpyCatcher Modified Epoxy Carriers

| | | Amount of | | Recovery |
|---|---|---|---|---|
| SpyCatcher | | Protein | Immobiliza- | Rate of |
| Modified | | Added | tion | Fluores- |
| Epoxy | Immobilization | (mg/g of | Efficiency | cence |
| Carrier | Method | carrier) | (%) | (%) |
| LX-1000EP | The present method | 10 | 77.6 | 75.6 |
| | Blocking control | 10 | 34.0 | 0.9 |
| | Epoxy method | 10 | 37.7 | 11.0 |
| LX-107S | The present method | 10 | 98.4 | 94.8 |
| | Blocking control | 10 | 98.9 | 95.3 |
| | Epoxy method | 10 | 100 | 97.2 |
| LX-103B | The present method | 10 | 63.3 | 72.6 |
| | Blocking control | 10 | 37.2 | 6.9 |
| | Epoxy method | 10 | 40.5 | 39.7 |
| HFA001 | The present method | 10 | 63.0 | 58.2 |
| | Blocking control | 10 | 11.5 | 0.5 |
| | Epoxy method | 10 | 11.9 | 0.6 |
| ECR8204 | The present method | 10 | 46.4 | 48.6 |
| | Blocking control | 10 | 59.9 | 59.1 |
| | Epoxy method | 10 | 77.3 | 75.9 |
| ECR8285 | The present method | 10 | 95.6 | 95.5 |
| | Blocking control | 10 | 97.6 | 97.2 |
| | Epoxy method | 10 | 98.6 | 97.2 |

### 5.5 Comparison on Immobilization Efficiency

### 5.5.1 Comparison on Immobilization Efficiency of Immobilized Protein of the Present Method and Epoxy Method

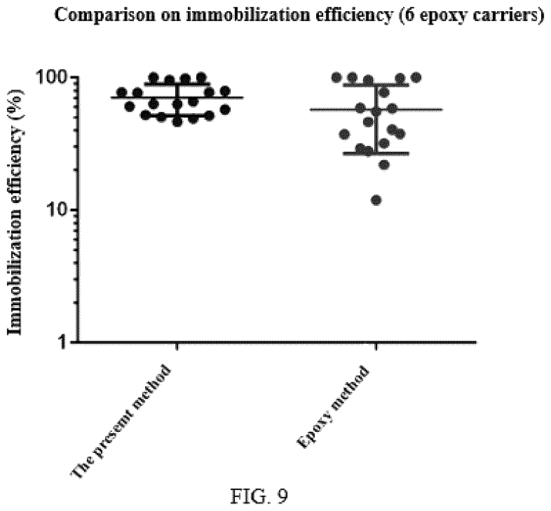

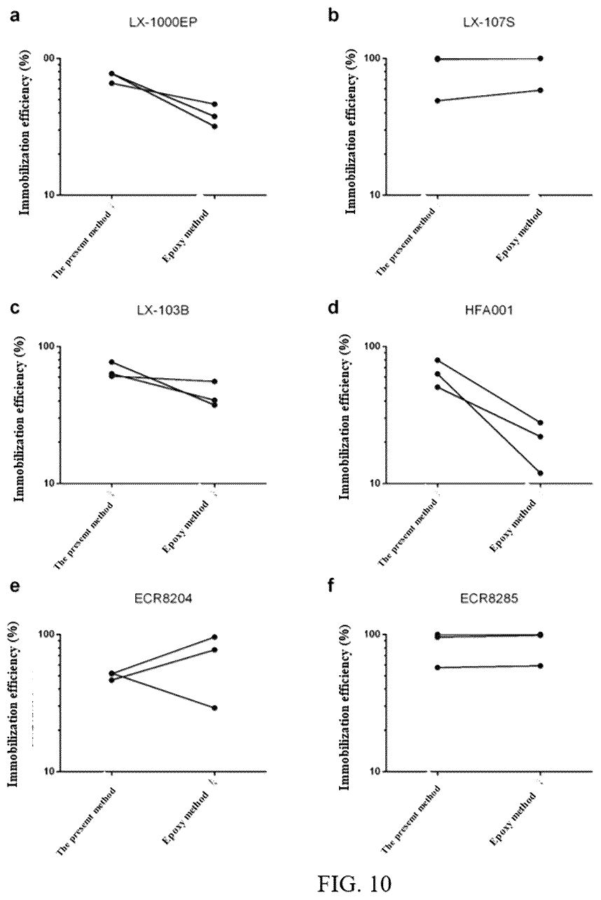

By the present method and the epoxy method, 6 epoxy carriers and 2 proteins (including purified protein SpyTag-RFP and a cell lysis solution containing target proteins SpyTag-RFP and SpyTag-GA) were selected for immobilization, and the results of the comparison on the immobilization efficiency were summarized as shown in FIG. 9. The statistical data of the immobilization efficiencies for the present method and the epoxy method were shown in Table 17. In the present method, an improved resin modified by SpyCatcher was used, and the present method exhibited a mean and median of the immobilization efficiency higher than the epoxy method, with a much lower variation coefficient than the epoxy method, indicating the present method exhibited higher immobilization efficiency and better homogeneity than the epoxy method.

TABLE 17
Statistical Data of Immobilization Efficiencies
for the Present Method and Epoxy Method

| Statistical Parameter | The Present Method | Epoxy method |
|---|---|---|
| Mean | 70.3% | 57.2% |
| Median | 64.6% | 51.0% |
| Variation coefficient | 26.5% | 53.4% |

5.5.2 Comparison on Immobilization Efficiency between Different Epoxy Carriers

The immobilization efficiencies of immobilizing proteins with a SpyCatcher modified resin obtained by the present method and an epoxy carrier were compared, with the results being as shown in FIG. 10. For most of epoxy carriers, the present method exhibited an immobilization efficiency which was significantly higher or not lower than that of the epoxy method.

### 5.6 Comparison on Recovery Rate of Activity

### 5.6.1 Comparison on Recovery Rate of Activity of Immobilizing Protein by the Present Method and Epoxy Method

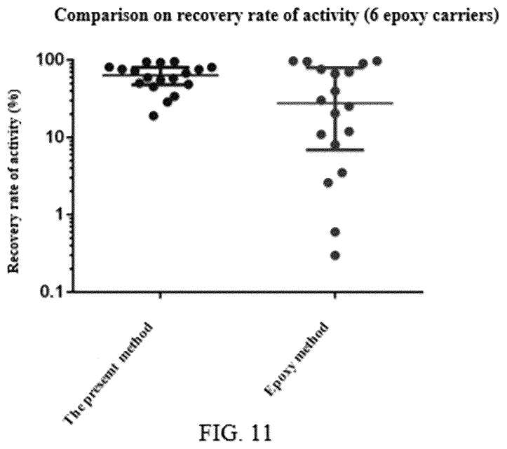

By the present method and the epoxy method, 6 epoxy carriers and 2 proteins (including purified protein SpyTag-RFP and a cell lysis solution containing target proteins SpyTag-RFP and SpyTag-GA) were selected for immobilization, and the results of the comparison on the recovery rate of activity were summarized as shown in FIG. 11. The statistical data of the immobilization efficiencies for the present method and the epoxy method were shown in Table 18. In the present method, an improved resin modified by SpyCatcher was used, and the present method exhibited a mean and median of the recovery rate of activity higher than the epoxy method, with a much lower range, interquartile range and variation coefficient than the epoxy method, indicating the present method exhibited higher immobilization efficiency and better homogeneity than the epoxy method. Thus, the method of the prior art exhibited unstable protein immobilization effect and was sensitive to the types of proteins and the types of the epoxy carriers. For a certain target protein, a lot of epoxy carrier screening work was required. In the present method, the epoxy carrier was improved so as to enhance the homogeneity of the carrier, and reduce the carrier screening work.

TABLE 18
Statistical Data of Immobilization Efficiencies
for the Present Method and Epoxy Method

| Statistical Parameter | The Present Method | Epoxy method |
|---|---|---|
| Mean | 63.4% | 27.8% |
| Median | 63.1% | 41.5% |
| Variation coefficient | 36.1% | 91.0% |

### 5.6.2 Comparison on Recovery Rate of Activity Between Different Epoxy Carriers

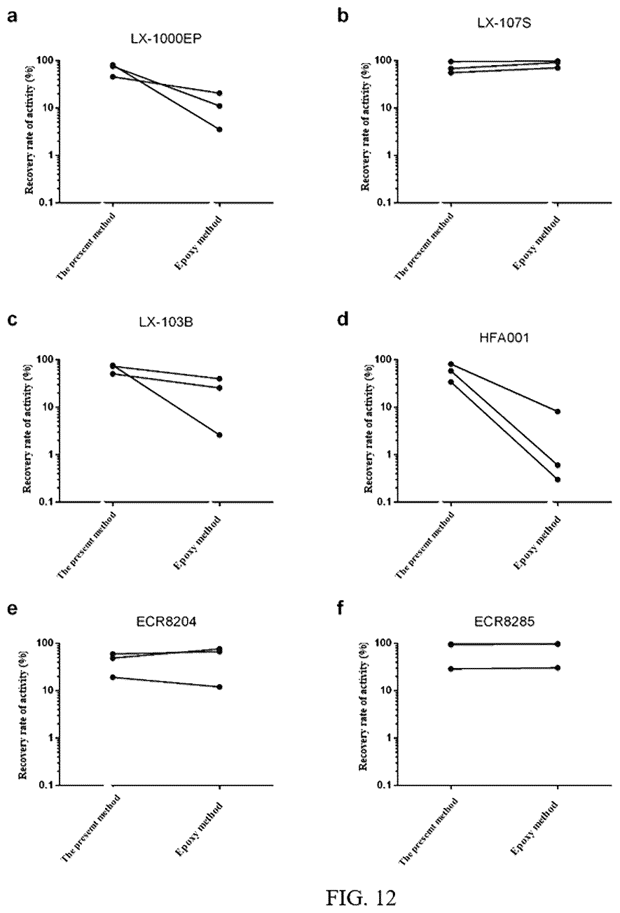

The immobilization efficiencies of immobilizing proteins with a SpyCatcher modified resin obtained by the present method and an epoxy carrier were compared, with the results being as shown in FIG. 12. For most of epoxy carriers, the present method exhibited a recovery rate of activity which was significantly higher or not lower than that of the epoxy method.

### 5.7 Comparison on Relative Activity

5.7.1 Comparison on Relative Activity of Immobilized Protein between Method of the Present Disclosure and Epoxy Method

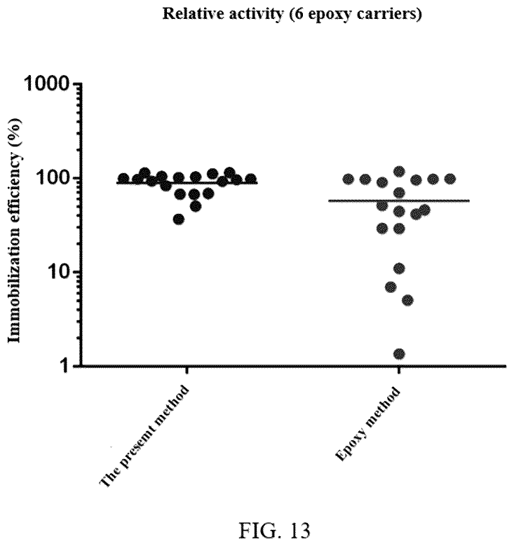

Based on the data of 5.5 and 5.6, the relative activity was calculated by the following formula: relative activity=recovery rate of activity/immobilization efficiency×100%. The results of the comparison on the relative activity of the immobilized protein between the present method and the epoxy method were summarized as shown in FIG. 13. The statistical data of the immobilization efficiencies for the present method and the epoxy method were shown in Table 19. The mean and the median of the relative activity of the protein immobilized with the SpyCatcher modified improved carrier in the present method were 89.1% and 96.9%, respectively, and there was almost no loss in the activity of the immobilized protein. However, the mean and the median of the relative activity of the protein immobilized by the epoxy method were 57.3% and 48.4%, respectively, and nearly half of the activity was lost. The variation coefficient of the relative activity for the immobilization by the present method was far below that for the epoxy method, indicating that the present method exhibited higher immobilization efficiency and better homogeneity than the epoxy method. The method of the prior art exhibited unstable protein immobilization effect and was sensitive to the types of proteins and the types of the epoxy carriers. For a certain target protein, a lot of epoxy carrier screening work was required. In the present method, the epoxy carrier was improved so as to enhance the homogeneity of the carrier, and reduce the carrier screening work.

TABLE 19
Statistical Data of Immobilization Efficiencies
for the Present Method and Epoxy Method

| Statistical Parameter | The Present Method | Epoxy method |
|---|---|---|
| Mean | 89.1% | 57.3% |
| Median | 96.9% | 48.4% |
| Variation coefficient | 25.2% | 68.1% |

5.7.2 Comparison on Relative Activity between Different Epoxy Carriers

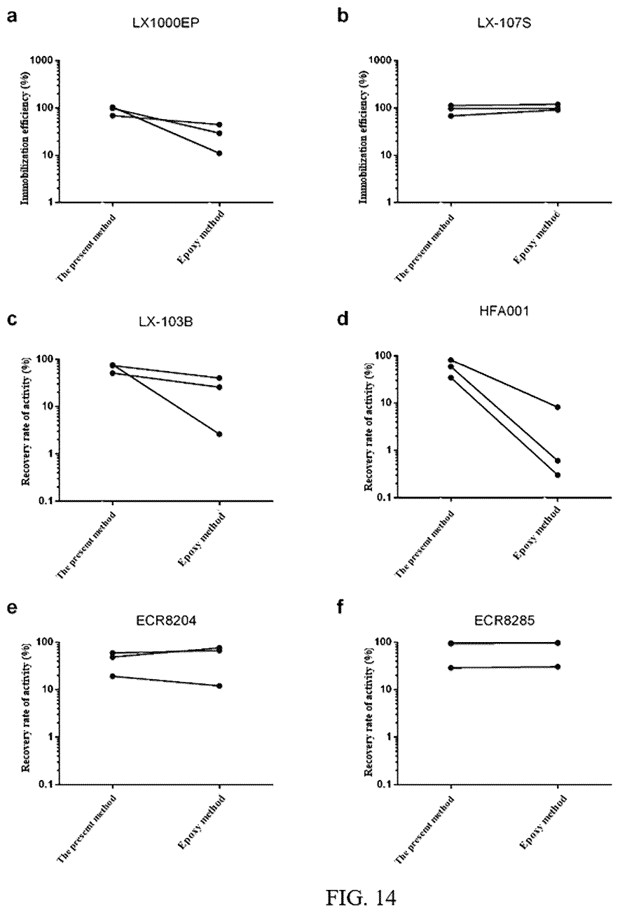

The relative activities of immobilizing proteins with the SpyCatcher modified carrier obtained by the present method and the epoxy carrier were compared, with the results being shown in FIG. 14. For most of epoxy carriers, the present method exhibited a relative activity which was significantly higher or not lower than that of the epoxy method.

### Example 6: Comparison of Immobilization Effects of Different Batches of Epoxy Carriers by the Present Method and an Existing Method

Stabilities of Different Batches (with immobilization conditions, immobilization efficiencies and recovery rates of activity being the same as those in Example 4.3)

SpyTag-RFP

| | Added Amount | Immobilization | Recovery Rate of |
|---|---|---|---|
| Batch | (mg/g of resin) | Efficiency (%) | Activity (%) |
| 1 | 15 | 60.3 | 41.2 |
| 2 | 15 | 67.8 | 57.3 |
| 3 | 15 | 65.9 | 45.2 |
| 4 | 15 | 62.1 | 67.1 |
| 5 | 15 | 70 | 49.7 |

Stabilities of Different Batches (with immobilization conditions, immobilization efficiencies and recovery rates of activity being the same as those in Example 4.3)

SpyTag-GA

| | Added Amount | Immobilization | Recovery Rate of |
|---|---|---|---|
| Batch | (mg/g of resin) | Efficiency (%) | Activity (%) |
| 1 | 15 | 94.7 | 93.4 |
| 2 | 15 | 92.2 | 94.3 |
| 3 | 15 | 90.9 | 81.6 |
| 4 | 15 | 86.6 | 82.7 |
| 5 | 15 | 96.9 | 91.7 |

### Example 7: Immobilization of SpyTag003-GA in Cell Lysis Solution with SpyCatcher003 Modified Carrier

Oligonucleotides and primers used in this example were shown in Table 20.

TABLE 20
Oligonucleotide Primer Sequences

| Oligonuc- | | SEQ |
|---|---|---|
| leotide/ | | ID |
| Primer | Nucleotide sequence (5′-3′)^{a} | NO |
| SpyTag003- | CTAGGCTCTAGAAATAATTTTGTTTAACTTT | 29 |
| GS-1 | AAGAAGG | |
| (SpyTag003-F) | | |
| SpyTag003- | TGCGGCACGCCGCGCATATGTATATCTCCTT | 30 |
| GS-2 | CTTAAAGTTAAACAAAATTATTTCTAGA | |
| SpyTag003- | GCGGCGTGCCGCATATTGTGATGGTGGATGC | 31 |
| GS-3 | GTATAAACGCTATAAAGGCGGTGGCGGC | |
| SpyTag003- | CGGTTCGAGCTCGCTTCCTCCCCCACCGCTT | 32 |
| GS-4 | CCCCCTCCTCCGCTGCCGCCACCGCCTT | |
| SpyTag003-R | ATACAAGTACACGGTTCGAGCTCGCTTCCT | 33 |

^{a}The underlined parts of the primer were recognition sites of restriction enzymes Xba I and Sac I.

### 7.1 Construction of Expression Plasmid for SpyCatcher003

The expression plasmid pET30a(+)-PTlinker-SpyCatcher003-His (SpyCatcher003, SEQ ID NO: 23) constructed in this example was used to express the protein PTlinker-SpyCatcher003-His, and the construction method was the same as the Example 1.1.

### 7.2 Construction of Expression Plasmid for SpyTag Fusion Protein

Construction of the plasmid pET30a(+)-SpyTag003-Gslinker-GA-His (SpyTag003, SEQ ID NO: 28): the codon optimized GA encoding sequence (SEQ ID NO: 16) of Pseudomonas sp. SY-77 was synthesized by Sangon Biotech (Shanghai) Co., Ltd., with the protein sequence of GA being of SEQ ID NO: 26. The oligonucleotide sequences SpyTag003-GS-1, 2, 3, and 4 as shown in Table 20 were used for PCR splicing and amplification to obtain the polynucleotide fragment Xba I-SpyTag003-Gslinker-Sac I, with the PCR splicing system being as shown in Table 21. Q5 high-fidelity DNA polymerase was used in the PCR reaction, and the PCR amplification system and procedure were shown in Table 22.

TABLE 21
PCR Splicing Reaction System of Oligonucleotide Sequences

| Reagent | Amount |
|---|---|
| SpyTag003-GS-1, 2, 3, and 4 (10 μM each) | 2 μL |
| 5 × Q5 Reaction buffer solution | 4 μL |
| Q5 DNA polymerase | 0.2 μL |
| 2.5 mM dNTPs | 4 μL |
| ddH₂O | Supplemented to 20 μL |

PCR splicing reaction procedure: a) 98° C. for 30 sec; b) 98° C. for 10 sec; c) 62° C. for 20 sec; d) 72° C. for 10 sec; e) 10 cycles of steps b)-d); f) 72° C. for 2 min; g) 10° C. hold.

TABLE 22
PCR Amplification Reaction System

| Reagent | Amount | |
|---|---|---|
| SpyTag003-F (10 μM) | 2.5 | μL |
| SpyTag003-R (10 μM) | 2.5 | μL |
| Xba I-SpyTag003-Gslinker-Sac I fragment | 1 | ng |
| 5 × Q5 Reaction buffer solution | 10 | μL |
| Q5 DNA polymerase | 0.5 | μL |
| 2.5 mM dNTPs | 4 | μL |

| | |
|---|---|
| ddH₂O | Supplemented to 50 μL |

PCR amplification reaction procedure: a) 98° C. for 30 sec; b) 98° C. for 10 sec; c) 66° C. for 20 sec; d) 72° C. for 10 sec; e) 30 cycles of steps b)-d); f) 72° C. for 2 min; g) 4° C. hold.

After the completion of the reaction, the PCR amplification product was detected by 2% agarose gel electrophoresis, and the result showed that a target DNA band as expected was obtained by PCR amplification. An ultra-thin DNA product purification kit was used to purify and recover the target DNA, which was preserved at −20° C. for later use.

The purified and recovered target DNA was doubly digested with restriction enzymes Xba I and Sac I and then linked to the plasmid pET30a(+)-SpyTag-Gslinker-GA-His which was doubly digested with the same enzymes. The linked product was transformed to competent cells of E. coli BL21(DE3). The transformed cells were spread onto an LB plate supplemented with 50 μg/mL of kanamycin (Kan) and positive clones were selected. The plasmid was extracted and sequenced, and the sequencing result indicated that the cloned pET30a(+)-SpyTag003-Gslinker-GA-His sequence was correct.

### 7.3 Expression, Purification, and Activity Determination of Fusion Proteins

The strain (containing the plasmid pET30a(+)-PTlinker-SpyCatcher003-His) constructed in Example 7.1 was inoculated to an LB liquid medium containing 50 μg/mL of kanamycin (Kan) in an inoculum amount of 1:50, cultured overnight, then transferred to a fresh LB medium (50 μg/mL of Kan) and cultured to the logarithmic phase (OD₆₀₀=0.4-0.6) in a shaker at 37° C., added with IPTG to a final concentration of 0.2 mM, and induced at 30° C. for 6 hours. The cell concentration OD₆₀₀ was measured, and cells were harvested.

The strain (containing the plasmid pET30a(+)-SpyTag003-GSlinker-GA-His) constructed in Example 7.2 was inoculated to an LB liquid medium containing 50 μg/mL of Kan in an inoculum amount of 1:50. The broth cultured overnight was transferred to a fresh LB medium (50 μg/mL of Kan) and cultured to the logarithmic phase (OD₆₀₀=0.4-0.6) in a shaker at 37° C., added with IPTG to a final concentration of 0.1 mM, and induced at 18° C. for 12 hours. The cell concentration OD₆₀₀ was measured, and cells were harvested.

The principles and methods of SDS-PAGE detection and quantitation, purification and dialysis of fusion proteins, and enzyme activity determination of fusion proteins were the same as those in Example 2.

### 7.4 Preparation of SpyCatcher003 Modified Carrier

The preparation method and the calculation of the immobilization efficiency were the same as those in Example 3.1, with the results being as shown in Table 23. When the amount of SpyCatcher003 added was 20 mg/g of carrier, the immobilization efficiency was 90.1%.

TABLE 23
Immobilization Results of SpyCatcher003

| | Immobilization Amount | Immobilization |
|---|---|---|
| Amount of Protein Added | of SpyCatcher | Efficiency |
| (mg/g of carrier) | (mg/g of carrier) | (%) |
| 20 | 18.0 | 90.1 |

The resulting immobilization SpyCatcher003-LX-1000EP was added to 12 times the volume of a blocking buffer solution (3 M glycine, pH 8.5) to react on a miniature rotator (Gilson, Roto-Mini Plus) at 25° C. and a speed of 25 rpm for 24 hours. After the completion of blocking, the blocking solution was removed by centrifugation, and the left was washed with PBS (0.1M, pH 7.0) for 3 times and preserved at 4° C.

7.5 Direct Immobilization of SpyTag003-GA from Cell Lysis Solution

The immobilization method and the calculation of the immobilization efficiency were the same as those in Example 4.3.4, with the results being as shown in Table 24. The blocking control exhibited the immobilization efficiency of SpyTag003-GA of 14.9%, while the epoxy method exhibited the immobilization efficiency of 31.9% and the present method exhibited the immobilization efficiency of 26.1%.

TABLE 24
Immobilization Results of SpyTag-GA in Cell Lysis Solution

| | Amount of | Immobilization |
|---|---|---|
| | Protein Added ^{a} | Efficiency |
| Immobilization Method | (mg/g of carrier) | (%) |
| Blocking control | 2.0 | 14.9 |
| Epoxy immobilization method | 2.0 | 31.9 |
| The present method | 2.0 | 26.1 |

^{a} The amount of the fusion protein added was estimated by SDS-PAGE.

### 7.6 Activity Detection of Immobilized SpyTag003-GA

The activity detection method and the calculation of the enzyme activity were the same as those in Example 4.4, with the results being as shown in Table 25. The epoxy method exhibited the recovery rate of activity of 33.1%, while the present method exhibited the recovery rate of activity of 23.8%.

TABLE 25
Results of Activity Detection of SpyTag003-GA

| Immobilization | Amount of Protein Added | Recovery Rate of |
|---|---|---|
| Method | (mg/g of carrier) | Enzyme Activityª (%) |
| Blocking | 2.0 | 25.9 |
| control | | |
| Epoxy | 2.0 | 33.1 |
| immobilization | | |
| method | | |
| The present | 2.0 | 23.8 |
| method | | |

${a}^{} Recovery rate of enzyme activity of immobilized GA ( \% ) = \frac{total enzyme activity of immobilized enzyme GA ( U / g resin )}{\begin{matrix}total enzyme activity in GA supernatant \\ before immobilization ( U / g resin )\end{matrix}} \times 100 \%$

The present invention provides a method of immobilizing an enzyme based on SpyCatcher/SpyTag reaction. There are many methods and ways to specifically implement the technical solutions, and the foregoing are merely exemplified embodiments of the present invention. A number of improvements and modifications can be made by a person of skilled in the art without departing from the principles of the present invention, and these improvements and modifications shall also be considered as falling within the protection scope of the present invention. The parts that are not explicitly defined in the examples can be implemented by the prior art.

1. A carrier modified by a SpyCatcher peptide, wherein the carrier, when not modified by the SpyCatcher peptide, comprises a group capable of reacting with an amino group (NH₂), wherein the SpyCatcher peptide is covalently attached to the carrier through reaction of an amino group with the group, and the SpyCatcher peptide linked to the carrier is capable of forming an isopeptide bond with a SpyTag peptide, preferably the group is selected from the group consisting of an epoxy group, an aldehyde group, an imide, a cyanate, an imidocarbonate and a hydrazide group.

2. The carrier modified by a SpyCatcher peptide according to claim 1, wherein the SpyCatcher peptide comprises the amino acid sequence as set forth in SEQ ID NO: 21, 22 or 23.

3. The carrier modified by a SpyCatcher peptide according to claim 1 or 2, wherein the carrier is an epoxy carrier or an amino carrier, preferably the carrier is made of a polyacrylate containing an epoxy group or amino group.

4. An epoxy resin carrier modified by a SpyCatcher peptide, wherein the SpyCatcher peptide is covalently attached to the carrier through reaction of an amino group with an epoxy group, preferably the epoxy resin carrier is made of a polyacrylate.

5. The epoxy resin carrier modified by a SpyCatcher peptide according to claim 4, wherein the particle size of the epoxy resin is about 100-1000 m, and/or the content of the epoxy group in the epoxy resin is about 50-800 μmol/g wet, preferably the particle size of the epoxy resin is about 100-350 μm, and/or the content of the epoxy group in the epoxy resin is about 50-100 μmol/g wet.

6. The carrier modified by a SpyCatcher peptide according to any one of claims 1 to 5, which is linked to a fusion protein comprising a SpyTag peptide and a target protein, wherein the SpyCatcher peptide forms an isopeptide bond with the SpyTag peptide, preferably the SpyTag peptide comprises the amino acid sequence of any one selected from the group consisting of SEQ ID NOs: 2, 11-15 and 28.

7. The carrier modified by a SpyCatcher peptide according to claim 6, wherein the target protein is an enzyme, preferably selected from the group consisting of glutaryl-7-amidocephalosporanic acid acylase, glucose isomerase, nitrile hydratase, penicillin amidase, aspartase, fumarase, amino-acylase, lactase, aspartate-β-decarboxylase and cephalosporin amidase.

8. A method of preparing a carrier modified by a SpyCatcher peptide, comprising:
(1) providing a carrier comprising a group capable of reacting with an amino group (NH₂), the group being preferably selected from the group consisting of an epoxy group, an aldehyde group, an imide, a cyanate, an imidocarbonate and a hydrazide group;
(2) bringing a SpyCatcher peptide into contact with the carrier in a liquid environment; optionally, activating the group before contact, and/or blocking the carrier after contact; and
(3) optionally, isolating the carrier modified by the SpyCatcher peptide.

9. The method according to claim 8, wherein the SpyCatcher peptide comprises the amino acid sequence as set forth in SEQ ID NO: 21, 22 or 23.

10. The method according to claim 8 or 9, wherein the carrier is an epoxy carrier or an amino carrier, preferably the carrier is made of a polyacrylate.

11. A method of immobilizing a protein, comprising:
(1) providing a carrier modified by a SpyCatcher peptide according to any one of claims 1 to 5 or a carrier modified by a SpyCatcher peptide obtained by the method according to any one of claims 8 to 10;
(2) providing a fusion protein comprising a SpyTag peptide and a target protein, preferably the SpyTag peptide comprises the amino acid sequence of any one selected from the group consisting of SEQ ID NOs: 2, 11-15 and 28;
(3) bringing the carrier in step (1) into contact with the fusion protein in step (2) under a condition of allowing the SpyCatcher peptide and the SpyTag peptide to form an isopeptide bond, and optionally, after contact, washing with a washing buffer to remove unimmobilized fusion protein; and
(4) optionally, isolating the carrier linked to the fusion protein.

12. The method according to claim 11, wherein step (1) comprises incubating a SpyCatcher peptide with a carrier not modified by a SpyCatcher peptide to obtain a carrier modified by the SpyCatcher peptide, and optionally, treating the carrier modified by the SpyCatcher peptide with a blocking buffer solution.

13. The method according to claim 11 or 12, wherein the target protein is an enzyme, preferably selected from the group consisting of glutaryl-7-amidocephalosporanic acid acylase, glucose isomerase, nitrile hydratase, penicillin amidase, aspartase, fumarase, amino-acylase, lactase, aspartate-β-decarboxylase, and cephalosporin amidase.

14. The method according to any one of claims 11 to 13, wherein the carrier is an epoxy resin or an amino carrier, preferably made of a polyacrylate.

15. A kit, comprising a carrier, a SpyCatcher peptide, and optionally a fusion protein of a SpyTag peptide and a target protein such as an enzyme, wherein the carrier and the SpyCatcher peptide are present separately or in the form of the carrier modified by the SpyCatcher peptide according to claim 1.

16. The kit according to claim 15, wherein the SpyCatcher peptide comprises the amino acid sequence as set forth in SEQ ID NO: 21, 22 or 23.

17. The kit according to claim 15 or 16, wherein the SpyTag peptide preferably comprises the amino acid sequence of any selected from the group consisting of SEQ ID NOs: 2, 11-15 and 28.

18. The kit according to any one of claims 15 to 17, wherein the target protein is an enzyme, preferably selected from the group consisting of glutaryl-7-amidocephalosporanic acid acylase, glucose isomerase, nitrile hydratase, penicillin amidase, aspartase, fumarase, amino-acylase, lactase, aspartate-β-decarboxylase, and cephalosporin amidase.

19. The kit according to any one of claims 15 to 18, wherein the carrier is an epoxy carrier or an amino carrier, preferably made of a polyacrylate.


SEQUENCE LISTING (33 sequences)

SEQ ID NO: 1 - protein, 138 aa, Artificial Sequence
  SpyCatcher
SYYHHHHHHDYDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 2 - protein, 13 aa, Artificial Sequence
  SpyTag
AHIVMVDAYKPTK

SEQ ID NO: 3 - DNA, 30 bp, Artificial Sequence
  SpyCatcher-F
attccatatgccgaccccaccgaccacgcc

SEQ ID NO: 4 - DNA, 45 bp, Artificial Sequence
  SpyCatcher-R
gtggtggtggtggtggtgctcgagaatgtgcgcatcgcctttggt

SEQ ID NO: 5 - DNA, 58 bp, Artificial Sequence
  RFP-F
atatacatatggcgcatattgtgatggtggatgcgtataaaccgaccaaaggcggtgg

SEQ ID NO: 6 - DNA, 32 bp, Artificial Sequence
  RFP-R
ggggggaagctttttatacagttcgtccatac

SEQ ID NO: 7 - DNA, 40 bp, Artificial Sequence
  GA-F
gcgagctcgaaccgaccagcaccccgcaggcgccgatcgc

SEQ ID NO: 8 - DNA, 35 bp, Artificial Sequence
  GA-R
gcaagcttcggtttaaagttaaacggggtgcgttc

SEQ ID NO: 9 - DNA, 5766 bp, Artificial Sequence
  pET-30a(+)-FS4-SpyCatcher DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgttcgaagcggaggttaaactgaaaatcgaactggaagttaaattcaaaccgaccccaccgaccacgccaacgccaccaaccaccccaaccccgacgccggaattcatgagctactatcaccaccaccaccaccacgactacgacatcccgaccaccgagaacctgtatttccagggtgcgatggtggacaccctgagcggcctgagcagcgaacagggtcaaagcggcgacatgaccatcgaggaagatagcgcgacccacattaagtttagcaaacgtgacgaggatggcaaggaactggcgggtgcgacgatggagctgcgtgatagcagcggtaaaaccatcagcacctggattagcgacggccaagtgaaggatttctacctgtatccgggtaaatacacctttgttgagaccgcggcgccggacggttatgaagtggcgaccgcgatcaccttcaccgttaacgaacagggtcaagtgaccgttaacggcaaggcgaccaaaggcgatgcgcacatttgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 10 - DNA, 6039 bp, Artificial Sequence
  pET-30a(+)-Spy-RFP DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggcgcatattgtgatggtggatgcgtataaaccgaccaaaggcggtggcggcagcggaggagggggaagcggtgggggaggaagcgagctcgtttctaaaggtgaagaagacaacatggctatcatcaaagaatttatgcgtttcaaagttcacatggaaggttctgtgaacggtcacgaatttgaaatcgaaggtgaaggtgaaggtcgtccgtatgaaggcacccagaccgctaaactgaaagttaccaaaggtggtccgctgccgttcgcttgggacatcctgtctccgcagttcatgtacggttctaaagcgtatgttaaacacccggctgacatcccggactacctgaaactgtctttcccggaaggtttcaaatgggaacgtgttatgaactttgaagacggtggtgttgttaccgttacccaggactcttctctgcaagacggtgaatttatctacaaagttaaactgcgtggcaccaacttcccgtctgacggtccggttatgcagaagaaaacgatgggttgggaagcgtcttctgaacgtatgtacccggaagacggtgctctgaaaggtgaaatcaaacagcgtctgaaactgaaagacggtggtcactacgacgctgaagttaaaaccacctacaaagctaaaaagccggttcaactgccgggtgcttacaacgtgaacatcaaactggacatcacctctcacaacgaagactacaccatcgttgaacagtacgaacgtgctgaaggtcgtcactctaccggcggtatggacgaactgtataaatgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - protein, 14 aa, Artificial Sequence
  SpyTag
VPTIVMVDAYKRYK

SEQ ID NO: 12 - protein, 13 aa, Artificial Sequence
  SpyTag
VPTIVMVDCRLDS

SEQ ID NO: 13 - protein, 13 aa, Artificial Sequence
  SpyTag
VPTIVMVDFWMRC

SEQ ID NO: 14 - protein, 13 aa, Artificial Sequence
  SpyTag
VPTIVMVDCCLFC

SEQ ID NO: 15 - protein, 13 aa, Artificial Sequence
  SpyTag
VPTIVMVDCYKRY

SEQ ID NO: 16 - DNA, 2043 bp, Artificial Sequence
  GA
atggaaccgaccagcaccccgcaggcgccgatcgcggcatataaaccgcgttctaacgaaattctgtgggatggctacggcgttccgcacatctacggcgttgacgcaccaagcgcgttctacggctacggttgggcacaggcccgttctcacggcgataacatcctgcgtctgtacggtgaagcgcgcggtaaaggcgcagaatattggggcccggattacgaacagacgacggtatggctgctgaccaacggcgttccagaacgtgctcagcagtggtacgctcagcagagcccggatttccgtgcgaacctggacgcatttgcggcgggcatcaacgcctacgcgcagcagaacccggacgatatttctccggaagttcgtcaggtactgccggtttctggtgcggacgtggtggcgcatgcacatcgtcttatgaacttcctgtatgtagccagccccggtcgtaccctgggcgaaggtgatccgcctgatcttgcggatcagggttccaacagctgggcggtggctccgggtaaaaccgctaacggcaacgcgctcctgctgcagaacccgcacttaagctggaccaccgattacttcacctactacgaagctcacctggttaccccggatttcgaaatctacggcgctacccagatcggtctcccggtgatccgtttcgcgttcaaccagcgtatgggtattaccaacaccgtgaacggcatggttggcgcaactaactaccgcctgaccctgcaagatggcggctacctgtatgatggtcaggtgcgtccgtttgaacgtccgcaggctagctaccgtctgcgtcaggccgacggtaccaccgttgacaaaccgctggaaatccgctcttccgtgcatggcccggtgtttgaacgtgcggatggtaccgcggtggcggttcgcgtggcgggcctggatcgtccgggcatgctggaacagtactttgatatgatcaccgcggactcgttcgatgactatgaagcggcgctggctcgtatgcaggttccgaccttcaacatcgtgtacgccgatcgtgaaggtaccattaactatagctttaatggcgtagctccgaaacgtgcggaaggcgatatcgcattctggcagggcctggttccgggtgatagcagccgctacctgtggaccgaaactcacccgctggatgatctgccgcgtgttaccaacccgccgggtggtttcgttcagaactctaacgacccgccttggaccccgacttggccggttacttataccccgaaagacttcccgagctatctggctccgcagaccccgcattcgctgcgcgcgcagcagtctgtacgcctgatgtccgaaaacgatgatctgaccctggaacgttttatggcgctgcagctgtctcaccgcgcggtgatggcggatcgcaccctgccggatctcatcccggctgcgctgattgacccggacccggaagttcaggccgccgcgcgtctgctggcagcgtgggaccgtgaattcacctccgactcccgtgcggcgctgctgtttgaagaatgggcgcgcctcttcgctggtcagaacttcgccggccaggcaggcttcgcgaccccgtggtctctggataaaccggtttctaccccgtacggcgttcgtgacccgaaggcggctgttgatcagctgcgtaccgcgattgcgaacaccaaacgcaaatacggtgccattgaccgtccgttcggcgacgcgtcacgcatgatcctgaacgatgtgaacgtgccgggtgcggccggctatggcaacctgggcagctttcgtgtgttcacctggtctgatccggatgaaaacggcgtccgtaccccggtgcatggtgaaacttgggtggctatgattgagttctctaccccggttcgtgcctacggtctgatgtcctacggcaactcccgtcagccgggcaccacccactactccgatcagatcgaacgtgtgtctcgtgctgattttcgtgaactgctgctgcgccgtgaacaggttgaagcataa

SEQ ID NO: 17 - DNA, 5715 bp, Artificial Sequence
  pET30a(+)-PT linker-SpyCatcher-His
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatgccgaccccaccgaccacgccaacgccaccaaccaccccaaccccgacgccggaattcatgagctactatcaccaccaccaccaccacgactacgacatcccgaccaccgagaacctgtatttccagggtgcgatggtggacaccctgagcggcctgagcagcgaacagggtcaaagcggcgacatgaccatcgaggaagatagcgcgacccacattaagtttagcaaacgtgacgaggatggcaaggaactggcgggtgcgacgatggagctgcgtgatagcagcggtaaaaccatcagcacctggattagcgacggccaagtgaaggatttctacctgtatccgggtaaatacacctttgttgagaccgcggcgccggacggttatgaagtggcgaccgcgatcaccttcaccgttaacgaacagggtcaagtgaccgttaacggcaaggcgaccaaaggcgatgcgcacattctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 18 - DNA, 6051 bp, Artificial Sequence
  pET30a(+)-SpyTag-GS linker-RFP-His
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggcgcatattgtgatggtggatgcgtataaaccgaccaaaggcggtggcggcagcggaggagggggaagcggtgggggaggaagcgagctcgtttctaaaggtgaagaagacaacatggctatcatcaaagaatttatgcgtttcaaagttcacatggaaggttctgtgaacggtcacgaatttgaaatcgaaggtgaaggtgaaggtcgtccgtatgaaggcacccagaccgctaaactgaaagttaccaaaggtggtccgctgccgttcgcttgggacatcctgtctccgcagttcatgtacggttctaaagcgtatgttaaacacccggctgacatcccggactacctgaaactgtctttcccggaaggtttcaaatgggaacgtgttatgaactttgaagacggtggtgttgttaccgttacccaggactcttctctgcaagacggtgaatttatctacaaagttaaactgcgtggcaccaacttcccgtctgacggtccggttatgcagaagaaaacgatgggttgggaagcgtcttctgaacgtatgtacccggaagacggtgctctgaaaggtgaaatcaaacagcgtctgaaactgaaagacggtggtcactacgacgctgaagttaaaaccacctacaaagctaaaaagccggttcaactgccgggtgcttacaacgtgaacatcaaactggacatcacctctcacaacgaagactacaccatcgttgaacagtacgaacgtgctgaaggtcgtcactctaccggcggtatggacgaactgtataaaaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 19 - DNA, 7419 bp, Artificial Sequence
  pET30a(+)-SpyTag-GSlinker-GA-His
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatggcgcatattgtgatggtggatgcgtataaaccgaccaaaggcggtggcggcagcggaggagggggaagcggtgggggaggaagcgagctcgaaccgaccagcaccccgcaggcgccgatcgcggcatataaaccgcgttctaacgaaattctgtgggatggctacggcgttccgcacatctacggcgttgacgcaccaagcgcgttctacggctacggttgggcacaggcccgttctcacggcgataacatcctgcgtctgtacggtgaagcgcgcggtaaaggcgcagaatattggggcccggattacgaacagacgacggtatggctgctgaccaacggcgttccagaacgtgctcagcagtggtacgctcagcagagcccggatttccgtgcgaacctggacgcatttgcggcgggcatcaacgcctacgcgcagcagaacccggacgatatttctccggaagttcgtcaggtactgccggtttctggtgcggacgtggtggcgcatgcacatcgtcttatgaacttcctgtatgtagccagccccggtcgtaccctgggcgaaggtgatccgcctgatcttgcggatcagggttccaacagctgggcggtggctccgggtaaaaccgctaacggcaacgcgctcctgctgcagaacccgcacttaagctggaccaccgattacttcacctactacgaagctcacctggttaccccggatttcgaaatctacggcgctacccagatcggtctcccggtgatccgtttcgcgttcaaccagcgtatgggtattaccaacaccgtgaacggcatggttggcgcaactaactaccgcctgaccctgcaagatggcggctacctgtatgatggtcaggtgcgtccgtttgaacgtccgcaggctagctaccgtctgcgtcaggccgacggtaccaccgttgacaaaccgctggaaatccgctcttccgtgcatggcccggtgtttgaacgtgcggatggtaccgcggtggcggttcgcgtggcgggcctggatcgtccgggcatgctggaacagtactttgatatgatcaccgcggactcgttcgatgactatgaagcggcgctggctcgtatgcaggttccgaccttcaacatcgtgtacgccgatcgtgaaggtaccattaactatagctttaatggcgtagctccgaaacgtgcggaaggcgatatcgcattctggcagggcctggttccgggtgatagcagccgctacctgtggaccgaaactcacccgctggatgatctgccgcgtgttaccaacccgccgggtggtttcgttcagaactctaacgacccgccttggaccccgacttggccggttacttataccccgaaagacttcccgagctatctggctccgcagaccccgcattcgctgcgcgcgcagcagtctgtacgcctgatgtccgaaaacgatgatctgaccctggaacgttttatggcgctgcagctgtctcaccgcgcggtgatggcggatcgcaccctgccggatctcatcccggctgcgctgattgacccggacccggaagttcaggccgccgcgcgtctgctggcagcgtgggaccgtgaattcacctccgactcccgtgcggcgctgctgtttgaagaatgggcgcgcctcttcgctggtcagaacttcgccggccaggcaggcttcgcgaccccgtggtctctggataaaccggtttctaccccgtacggcgttcgtgacccgaaggcggctgttgatcagctgcgtaccgcgattgcgaacaccaaacgcaaatacggtgccattgaccgtccgttcggcgacgcgtcacgcatgatcctgaacgatgtgaacgtgccgggtgcggccggctatggcaacctgggcagctttcgtgtgttcacctggtctgatccggatgaaaacggcgtccgtaccccggtgcatggtgaaacttgggtggctatgattgagttctctaccccggttcgtgcctacggtctgatgtcctacggcaactcccgtcagccgggcaccacccactactccgatcagatcgaacgtgtgtctcgtgctgattttcgtgaactgctgctgcgccgtgaacaggttgaagcagctgttcaggaacgcaccccgtttaactttaaaccgaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 20 - protein, 113 aa, Artificial Sequence
  Spycatcher
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 21 - protein, 91 aa, Artificial Sequence
  Spycatcher
SATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 22 - protein, 113 aa, Artificial Sequence
  Spycatcher
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 23 - protein, 113 aa, Artificial Sequence
  Spycatcher
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 24 - protein, 265 aa, Artificial Sequence
  SpyTag-RFP
AHIVMVDAYKPTKGGGGSGGGGSGGGGSELVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 25 - protein, 721 aa, Artificial Sequence
  SpyTag-GA
AHIVMVDAYKPTKGGGGSGGGGSGGGGSELEPTSTPQAPIAAYKPRSNEILWDGYGVPHIYGVDAPSAFYGYGWAQARSHGDNILRLYGEARGKGAEYWGPDYEQTTVWLLTNGVPERAQQWYAQQSPDFRANLDAFAAGINAYAQQNPDDISPEVRQVLPVSGADVVAHAHRLMNFLYVASPGRTLGEGDPPDLADQGSNSWAVAPGKTANGNALLLQNPHLSWTTDYFTYYEAHLVTPDFEIYGATQIGLPVIRFAFNQRMGITNTVNGMVGATNYRLTLQDGGYLYDGQVRPFERPQASYRLRQADGTTVDKPLEIRSSVHGPVFERADGTAVAVRVAGLDRPGMLEQYFDMITADSFDDYEAALARMQVPTFNIVYADREGTINYSFNGVAPKRAEGDIAFWQGLVPGDSSRYLWTETHPLDDLPRVTNPPGGFVQNSNDPPWTPTWPVTYTPKDFPSYLAPQTPHSLRAQQSVRLMSENDDLTLERFMALQLSHRAVMADRTLPDLIPAALIDPDPEVQAAARLLAAWDREFTSDSRAALLFEEWARLFAGQNFAGQAGFATPWSLDKPVSTPYGVRDPKAAVDQLRTAIANTKRKYGAIDRPFGDASRMILNDVNVPGAAGYGNLGSFRVFTWSDPDENGVRTPVHGETWVAMIEFSTPVRAYGLMSYGNSRQPGTTHYSDQIERVSRADFRELLLRREQVEAAVQERTPFNFKP

SEQ ID NO: 26 - protein, 691 aa, Pseudomonas sp. SY-77
EPTSTPQAPIAAYKPRSNEILWDGYGVPHIYGVDAPSAFYGYGWAQARSHGDNILRLYGEARGKGAEYWGPDYEQTTVWLLTNGVPERAQQWYAQQSPDFRANLDAFAAGINAYAQQNPDDISPEVRQVLPVSGADVVAHAHRLMNFLYVASPGRTLGEGDPPDLADQGSNSWAVAPGKTANGNALLLQNPHLSWTTDYFTYYEAHLVTPDFEIYGATQIGLPVIRFAFNQRMGITNTVNGMVGATNYRLTLQDGGYLYDGQVRPFERPQASYRLRQADGTTVDKPLEIRSSVHGPVFERADGTAVAVRVAGLDRPGMLEQYFDMITADSFDDYEAALARMQVPTFNIVYADREGTINYSFNGVAPKRAEGDIAFWQGLVPGDSSRYLWTETHPLDDLPRVTNPPGGFVQNSNDPPWTPTWPVTYTPKDFPSYLAPQTPHSLRAQQSVRLMSENDDLTLERFMALQLSHRAVMADRTLPDLIPAALIDPDPEVQAAARLLAAWDREFTSDSRAALLFEEWARLFAGQNFAGQAGFATPWSLDKPVSTPYGVRDPKAAVDQLRTAIANTKRKYGAIDRPFGDASRMILNDVNVPGAAGYGNLGSFRVFTWSDPDENGVRTPVHGETWVAMIEFSTPVRAYGLMSYGNSRQPGTTHYSDQIERVSRADFRELLLRREQVEAAVQERTPFNFKP

SEQ ID NO: 27 - protein, 15 aa, Artificial Sequence
  linker
GGGGSGGGGSGGGGS

SEQ ID NO: 28 - protein, 16 aa, Artificial Sequence
  spytag
RGVPHIVMVDAYKRYK

SEQ ID NO: 29 - DNA, 38 bp, Artificial Sequence
  SpyTag003-GS-1/SpyTag003-F
ctaggctctagaaataattttgtttaactttaagaagg

SEQ ID NO: 30 - DNA, 59 bp, Artificial Sequence
  SpyTag003-GS-2
tgcggcacgccgcgcatatgtatatctccttcttaaagttaaacaaaattatttctaga

SEQ ID NO: 31 - DNA, 59 bp, Artificial Sequence
  SpyTag003-GS-3
gcggcgtgccgcatattgtgatggtggatgcgtataaacgctataaaggcggtggcggc

SEQ ID NO: 32 - DNA, 59 bp, Artificial Sequence
  SpyTag003-GS-4
cggttcgagctcgcttcctcccccaccgcttccccctcctccgctgccgccaccgcctt

SEQ ID NO: 33 - DNA, 30 bp, Artificial Sequence
  SpyTag003-R
atacaagtacacggttcgagctcgcttcct